(19) United States
(12) Patent Application Publication
(10) Pub. No.: US 2017/0189095 A1
(43) Pub. Date: Jul. 6, 2017

(54) MULTIPLE PORT ELECTRICAL ISOLATION TECHNIQUE FOR SURGICAL INSTRUMENTS

(71) Applicant: Ethicon Endo-Surgery, LLC, Guaynabo, PR (US)

(72) Inventors: Benjamin J. Danziger, Cincinnati, OH (US); David C. Yates, West Chester, OH (US); Jeffrey L. Aldridge, Lebanon, OH (US); Chris Dums, Neenah, WI (US); Joseph Scholler, Louisville, CO (US); Douglas Patrick Sterlina, Durham, NC (US); Jonathan Michael Bonte, Bartlein, CT (US)

(21) Appl. No.: 14/986,336

(22) Filed: Dec. 31, 2015

(51) Int. Cl. A61B 18/12; A61B 18/14

(52) U.S. Cl. CPC A61B 18/1206; A61B 18/1445; A61B 2018/00779

(57) ABSTRACT

Various forms are directed to systems and methods for operating a surgical instrument that includes a generator configured to provide an energy signal for treating tissue. The generator includes one or more ports for delivering the energy signal to one or more surgical instruments configured to treat tissue, and the one or more surgical instruments are electrically coupled to the respective one or more ports simultaneously. The energy signal includes an energy component comprising one or more energy modalities that is delivered through the one or more ports. The one or more energy modalities includes ultrasonic, bipolar radio frequency (RF), monopolar RF, reversible electroporation, irreversible electroporation, or microwave component, or any combination thereof.

### CROSS-REFERENCE TO RELATED APPLICATIONS

This application is related to U.S. Patent Application referenced under Attorney Docket No. END7754USNP/150455 and titled ADAPTER FOR ELECTRICAL SURGICAL INSTRUMENTS, the disclosure of which is herein incorporated by reference in its entirety.

### TECHNICAL FIELD

The present disclosure generally relates to surgical systems and, more particularly, to electrical surgical systems that enable surgical procedures and adapt and customize algorithms for performing such procedures based on the type of tissue being treated. Still more particularly, the present disclosure relates to surgical systems that employ instruments and generators utilizing ultrasonic, bipolar or monopolar radio frequency (RF), irreversible and/or reversible electroporation, and/or microwave technologies, among others.

### BACKGROUND

Ultrasonic surgical instruments are finding increasingly widespread applications in surgical procedures by virtue of the unique performance characteristics of such instruments. Depending upon specific instrument configurations and operational parameters, ultrasonic surgical instruments can provide substantially simultaneous cutting of tissue and hemostasis by coagulation, desirably minimizing patient trauma. The cutting action is typically realized by an-end effector, or blade tip, at the distal end of the instrument, which transmits ultrasonic energy to tissue brought into contact with the end effector. Ultrasonic instruments of this nature can be configured for open surgical use, laparoscopic, or endoscopic surgical procedures including robotic-assisted procedures.

Some surgical instruments utilize ultrasonic energy for precise cutting and controlled coagulation. Ultrasonic energy cuts and coagulates by vibrating a blade in contact with tissue. Vibrating at high frequencies (e.g., 55,500 times per second), the ultrasonic blade denatures protein in the tissue to form a sticky coagulum. Pressure exerted on tissue with the blade surface collapses blood vessels and allows the coagulum to form a hemostatic seal. The precision of cutting and coagulation is controlled by the surgeon's technique and adjusting the power level, blade edge, tissue traction, and blade pressure.

Electrosurgical devices for applying electrical energy to tissue in order to treat and/or destroy the tissue are also finding increasingly widespread applications in surgical procedures. An electrosurgical device typically includes a hand piece, an instrument having a distally-mounted end effector (e.g., one or more electrodes). The end effector can be positioned against the tissue such that electrical current is introduced into the tissue. Electrosurgical devices can be configured for bipolar or monopolar operation. During bipolar operation, current is introduced into and returned from the tissue by active and return electrodes, respectively, of the end effector. During monopolar operation, current is introduced into the tissue by an active electrode of the end effector and returned through a return electrode (e.g., a grounding pad) separately located on a patient's body. Heat generated by the current flowing through the tissue may form hemostatic seals within the tissue and/or between tissues and thus may be particularly useful for sealing blood vessels, for example. The end effector of an electrosurgical device also may include a cutting member that is movable relative to the tissue and the electrodes to transect the tissue.

Electrical energy applied by an electrosurgical device can be transmitted to the instrument by a generator in communication with the hand piece. The electrical energy may be in the form of RF energy that may be in a frequency range described in EN 60601-2-2:2009+A11:2011, Definition 201.3.218—HIGH FREQUENCY. For example, the frequencies in monopolar RF applications are typically restricted to less than 5 MHz. However, in bipolar RF applications, the frequency can be almost anything. Frequencies above 200 kHz can be typically used for MONOPOLAR applications in order to avoid the unwanted stimulation of nerves and muscles which would result from the use of low frequency current. Lower frequencies may be used for BIPOLAR techniques if the RISK ANALYSIS shows the possibility of neuromuscular stimulation has been mitigated to an acceptable level. Normally, frequencies above 5 MHz are not used in order to minimize the problems associated with HIGH FREQUENCY LEAKAGE CURRENTS. However, higher frequencies may be used in the case of BIPOLAR techniques. It is generally recognized that 10 mA is the lower threshold of thermal effects on tissue.

In application, an electrosurgical device can transmit low frequency RF energy through tissue, which causes ionic agitation, or friction, in effect resistive heating, thereby increasing the temperature of the tissue. Because a sharp boundary is created between the affected tissue and the surrounding tissue, surgeons can operate with a high level of precision and control, without sacrificing un-targeted adjacent tissue. The low operating temperatures of RF energy is useful for removing, shrinking, or sculpting soft tissue while simultaneously sealing blood vessels. RF energy works particularly well on connective tissue, which is primarily comprised of collagen and shrinks when contacted by heat.

Other electrical surgical instruments include, without limitation, irreversible and/or reversible electroporation, and/or microwave technologies, among others. Accordingly, the techniques disclosed herein are applicable to ultrasonic, bipolar or monopolar RF (electrosurgical), irreversible and/or reversible electroporation, and/or microwave based surgical instruments, among others.

A challenge of using these medical devices is the inability to control and customize the power output depending on the type of tissue being treated by the devices. Surgical generators described herein are configured to provide auxiliary power to electrical surgical instruments. An adapter provides electrical power to these electrical surgical instruments to run motors, sample, and communicate sensor data, provide indicators to the surgeon, etc. Other energy modalities such as reversible and/or irreversible electroporation and microwave energy, can benefit from the techniques disclosed herein. It would be desirable to provide a surgical instrument that overcomes some of the deficiencies of current instruments. The surgical systems described herein overcome those deficiencies.

### BRIEF DESCRIPTION OF THE FIGURES

The novel features of the described forms are set forth with particularity in the appended claims. The described forms, however, both as to organization and methods of operation, may be best understood by reference to the following description, taken in conjunction with the accompanying drawings in which:

FIG. 1 illustrates one form of a surgical system comprising a generator and various surgical instruments usable therewith;

FIG. 2 is a diagram of the ultrasonic surgical instrument of FIG. 1;

FIG. 3 is a diagram of the surgical system of FIG. 1;

FIG. 4 is a model illustrating motional branch current in one form;

FIG. 5 is a structural view of a generator architecture in one form;

FIG. 6 illustrates one form of a drive system of a generator, which creates the ultrasonic electrical signal for driving an ultrasonic transducer;

FIG. 7 illustrates one form of a drive system of a generator comprising a tissue impedance module;

FIG. 8 illustrates an example of a generator for delivering multiple energy modalities to a surgical instrument;

FIG. 9 is a diagram of a system for delivering multiple energy modalities to a plurality of surgical instruments;

FIG. 10 illustrates a communications architecture of a system for delivering multiple energy modalities to a plurality of surgical instruments;

FIG. 11 illustrates a communications architecture of a system for delivering multiple energy modalities to a plurality of surgical instruments;

FIG. 12 illustrates a communications architecture of a system for delivering multiple energy modalities to a plurality of surgical instruments;

FIG. 13 is an example graph of two waveforms of energy modalities from a generator;

FIG. 14 is an example graph of the sum of the waveforms of FIG. 13;

FIG. 15 is an example graph of sum of the waveforms of FIG. 13 with a one energy modality waveform dependent on another energy modality waveform;

FIG. 16 is an example graph of the sum of the waveforms of FIG. 13 with the one energy modality waveform being a function of another energy modality waveform;

FIG. 17 is an example graph of a complex modality energy waveform;

FIG. 18 illustrates one form of a surgical system comprising an adapter for coupling to a generator and various surgical instruments;

FIG. 19 illustrates example relays for use with an adapter;

FIG. 20 illustrates an example aspect of energy storage circuit of the adapter of FIG. 18;

FIG. 21 illustrates an example AC/DC converter of the adapter of FIG. 18;

FIG. 22 illustrates one aspect of an identification scheme for use with an adapter;

FIG. 23 illustrates another aspect of an identification scheme for use with an adapter;

FIG. 24 illustrates an example adapter and a path for a charging current to return to ground;

FIG. 25 illustrates an example adapter including a supply for separating various grounds; and

FIG. 26 is an example graph of capacitance and Equivalent Series Resistance (ESR) of an adapter.

### DESCRIPTION

Before explaining various forms of ultrasonic surgical instruments in detail, it should be noted that the illustrative forms are not limited in application or use to the details of construction and arrangement of parts illustrated in the accompanying drawings and description. The illustrative forms may be implemented or incorporated in other forms, variations and modifications, and may be practiced or carried out in various ways. Further, unless otherwise indicated, the terms and expressions employed herein have been chosen for the purpose of describing the illustrative forms for the convenience of the reader and are not for the purpose of limitation thereof.

Further, it is understood that any one or more of the following-described forms, expressions of forms, examples, can be combined with any one or more of the other following-described forms, expressions of forms, and examples.

Various forms are directed to improved ultrasonic surgical instruments configured for effecting tissue dissecting, cutting, and/or coagulation during surgical procedures. In one form, an ultrasonic surgical instrument apparatus is configured for use in open surgical procedures, but has applications in other types of surgery, such as laparoscopic, endoscopic, and robotic-assisted procedures. Versatile use is facilitated by selective use of ultrasonic energy.

The various forms will be described in combination with an ultrasonic instrument as described herein. Such description is provided by way of example, and not limitation, and is not intended to limit the scope and applications thereof. For example, any one of the described forms is useful in combination with a multitude of ultrasonic instruments including those described in, for example, U.S. Pat. Nos. 5,938,633; 5,935,144; 5,944,737; 5,322,055; 5,630,420; and 5,449,370, which are each incorporated by reference herein in their entirety.

As will become apparent from the following description, it is contemplated that forms of the surgical instrument described herein may be used in association with an oscillator unit of a surgical system, whereby ultrasonic energy from the oscillator unit provides the ultrasonic actuation for the present surgical instrument. It is also contemplated that forms of the surgical instrument described herein may be used in association with a signal generator unit of a surgical system, whereby electrical energy in the form of RF, for example, is used to provide feedback to the user regarding the surgical instrument. The ultrasonic oscillator and/or the signal generator unit may be non-detachably integrated with the surgical instrument or may be provided as separate components, which can be electrically attachable to the surgical instrument.

One form of the present surgical apparatus is particularly configured for disposable use by virtue of its straightforward construction. However, it is also contemplated that other forms of the present surgical instrument can be configured for non-disposable or multiple uses. Detachable connection of the present surgical instrument with an associated oscillator and signal generator unit is presently disclosed for single-patient use for illustrative purposes. However, non-detachable integrated connection of the present surgical instrument with an associated oscillator and/or signal generator unit is also contemplated. Accordingly, various forms of the presently described surgical instruments may be configured for single use and/or multiple use with either detachable and/or non-detachable integral oscillator and/or signal generator unit, without limitation, and all combinations of such configurations are contemplated to be within the scope of the present disclosure.

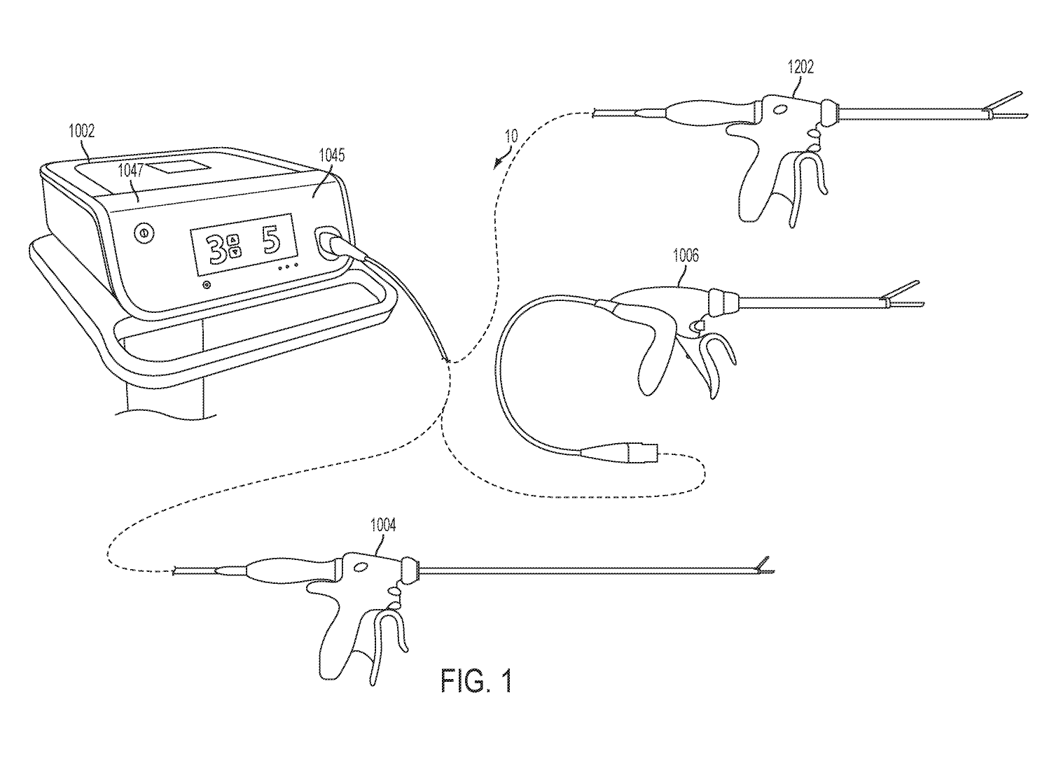

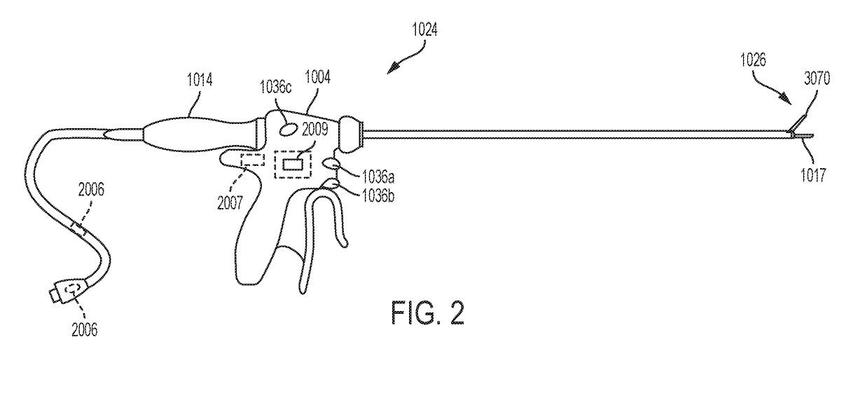

With reference to FIGS. 1-5, one form of a surgical system 10 including an ultrasonic surgical instrument is illustrated. FIG. 1 illustrates one form of a surgical system 10 comprising a generator 1002 and various surgical instruments 1004, 1006, 1202 usable therewith. FIG. 2 is a diagram of the ultrasonic surgical instrument 1004 of FIG. 1. With reference to both FIGS. 1 and 2, the generator 1002 is configurable for use with a variety of surgical devices. According to various forms, the generator 1002 may be configurable for use with different surgical devices of different types including, for example, the ultrasonic surgical instrument 1004, electrosurgical or RF surgical devices, such as, the electrosurgical instrument 1006, and multifunction devices 1202 that integrate electrosurgical RF and ultrasonic energies delivered simultaneously from the generator 1002. Although in the form of FIG. 1, the generator 1002 is shown separate from the surgical instruments 1004, 1006, 1202 in one form, the generator 1002 may be formed integrally with either of the surgical instruments 1004, 1006, 1202 to form a unitary surgical system. The generator 1002 comprises an input device 1045 located on a front panel of the generator 1002 console. The input device 1045 may comprise any suitable device that generates signals suitable for programming the operation of the generator 1002.

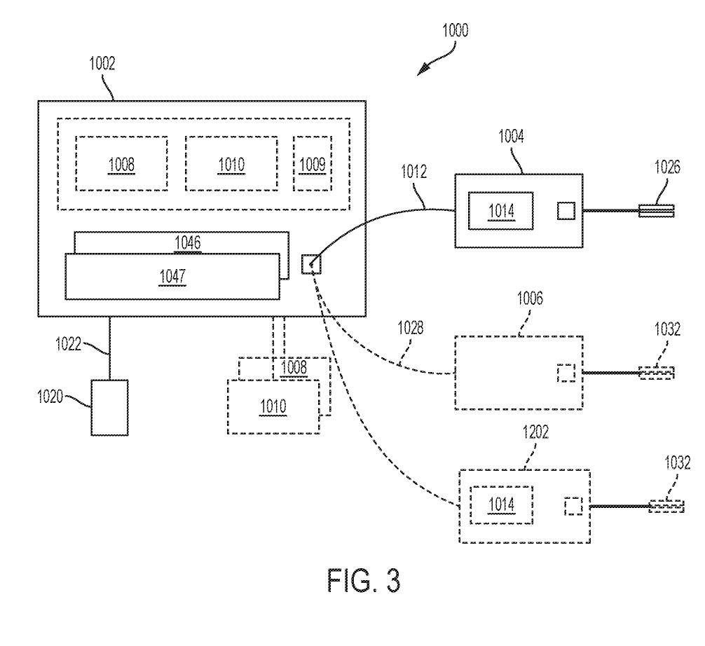

FIG. 3 is a diagram of the surgical system 10 of FIG. 1. In various forms, the generator 1002 may comprise several separate functional elements, such as modules and/or blocks. Different functional elements or modules may be configured for driving the different kinds of surgical instruments 1004, 1006, 1202. For example, an ultrasonic generator module 1008 may drive ultrasonic devices such as the ultrasonic surgical instrument 1004. An electrosurgery/RF generator module 1010 may drive the electrosurgical instrument 1006. For example, the respective modules 1008, 1010 may generate respective drive signals for driving the surgical instruments 1004, 1006, 1202. In various forms, the ultrasonic generator module 1008 and/or the electrosurgery/RF generator module 1010 may be formed integrally with the generator 1002. Alternatively, one or more of the modules 1008, 1010 may be provided as a separate circuit module electrically coupled to the generator 1002. (The modules 1008 and 1010 are shown in phantom to illustrate this option.) Also, in some forms, the electrosurgery/RF generator module 1010 may be formed integrally with the ultrasonic generator module 1008, or vice versa. Also, in some forms, the generator 1002 may be omitted entirely and the modules 1008, 1010 may be executed by processors or other hardware within the respective surgical instruments 1004, 1006, 1202.

In other forms, the electrical outputs of the ultrasonic generator module 1008 and the electrosurgery/RF generator module 1010 may be combined into at least one electrical signal capable of driving the multifunction device 1202 simultaneously with electrosurgical RF and ultrasonic energies. The multifunction device 1202 comprises an ultrasonic transducer 1014 coupled to an ultrasonic blade and one or more electrodes in the end effector 1032 to receive electrosurgical RF energy. In such implementations, the combined RF/ultrasonic signal is coupled to the multifunction device 1202. The multifunction device 1202 comprises signal processing components to split the combined RF/ultrasonic signal such that the RF signal can be delivered to the electrodes in the end effector 1032 and the ultrasonic signal can be delivered to the ultrasonic transducer 1014.

In accordance with the described forms, the ultrasonic generator module 1008 may produce a drive signal or signals of particular voltages, currents, and frequencies, e.g., 55,500 cycles per second (Hz). The drive signal or signals may be provided to the ultrasonic surgical instrument 1004, and specifically to the ultrasonic transducer 1014, which may operate, for example, as described above. The ultrasonic transducer 1014 and a waveguide extending through the shaft (waveguide not shown in FIG. 2) may collectively form an ultrasonic drive system driving an ultrasonic blade 1017 of an end effector 1026. In one form, the generator 1002 may be configured to produce a drive signal of a particular voltage, current, and/or frequency output signal that can be stepped or otherwise modified with high resolution, accuracy, and repeatability.

The generator 1002 may be activated to provide the drive signal to the ultrasonic transducer 1014 in any suitable manner. For example, the generator 1002 may comprise a foot switch 1020 coupled to the generator 1002 via a footswitch cable 1022. A clinician may activate the ultrasonic transducer 1014 by depressing the foot switch 1020. In addition, or instead of the foot switch 1020 some forms of the ultrasonic surgical instrument 1004 may utilize one or more switches positioned on the hand piece that, when activated, may cause the generator 1002 to activate the ultrasonic transducer 1014. In one form, for example, the one or more switches may comprise a pair of toggle buttons 1036a, 1036b (FIG. 2), for example, to determine an operating mode of the ultrasonic surgical instrument 1004. When the toggle button 1036a is depressed, for example, the generator 1002 may provide a maximum drive signal to the ultrasonic transducer 1014, causing it to produce maximum ultrasonic energy output. Depressing toggle button 1036b may cause the generator 1002 to provide a user-selectable drive signal to the ultrasonic transducer 1014, causing it to produce less than the maximum ultrasonic energy output. The ultrasonic surgical instrument 1004 additionally or alternatively may comprise a second switch (not shown) to, for example, indicate a position of a jaw closure trigger for operating jaws of the end effector 1026. Also, in some forms, the generator 1002 may be activated based on the position of the jaw closure trigger, (e.g., as the clinician depresses the jaw closure trigger to close the jaws, ultrasonic energy may be applied).

Additionally or alternatively, the one or more switches may comprises a toggle button 1036c that, when depressed, causes the generator 1002 to provide a pulsed output. The pulses may be provided at any suitable frequency and grouping, for example. In certain forms, the power level of the pulses may be the power levels associated with toggle buttons 1036a, 1036b (maximum, less than maximum), for example.

It will be appreciated that a ultrasonic surgical instrument 1004 may comprise any combination of the toggle buttons 1036a, 1036b, 1036c. For example, the ultrasonic surgical instrument 1004 could be configured with two toggle buttons: a toggle button 1036a for producing maximum ultrasonic energy output and a toggle button 1036c for producing a pulsed output at either the maximum or less than maximum power level. In this way, the drive signal output configuration of the generator 1002 could be 5 continuous signals and 5 or 4 or 3 or 2 or 1 pulsed signals. In certain forms, the specific drive signal configuration may be controlled based upon, for example, a non-volatile memory (NVM) such as an electrically erasable programmable read only memory (EEPROM) settings in the generator 1002 and/or user power level selection(s).

In certain forms, a two-position switch may be provided as an alternative to a toggle button 1036c. For example, a ultrasonic surgical instrument 1004 may include a toggle button 1036a for producing a continuous output at a maximum power level and a two-position toggle button 1036b. In a first detented position, toggle button 1036b may produce a continuous output at a less than maximum power level, and in a second detented position the toggle button 1036b may produce a pulsed output (e.g., at either a maximum or less than maximum power level, depending upon the NVM settings).

In accordance with the described forms, the electrosurgery/RF generator module 1010 may generate a drive signal or signals with output power to perform bipolar electrosurgery using RF energy. In bipolar electrosurgery applications, the drive signal may be provided, for example, to electrodes of the electrosurgical instrument 1006, for example. Accordingly, the generator 1002 may be configured for therapeutic purposes by applying electrical energy to the tissue for treating the tissue (e.g., coagulation, cauterization, tissue welding).

The generator 1002 may comprise an input device 1045 (FIG. 1) located, for example, on a front panel of the generator 1002 console. The input device 1045 may comprise any suitable device that generates signals suitable for programming the operation of the generator 1002. In operation, the user can program or otherwise control operation of the generator 1002 using the input device 1045. The input device 1045 may comprise any suitable device that generates signals that can be used by the generator (e.g., by one or more processors contained in the generator) to control the operation of the generator 1002 (e.g., operation of the ultrasonic generator module 1008 and/or electrosurgery/RF generator module 1010). In various forms, the input device 1045 includes one or more of buttons, switches, thumbwheels, keyboard, keypad, touch screen monitor, pointing device, remote connection to a general purpose or dedicated computer. In other forms, the input device 1045 may comprise a suitable user interface, such as one or more user interface screens displayed on a touch screen monitor, for example. Accordingly, by way of the input device 1045, the user can set or program various operating parameters of the generator, such as, for example, current (I), voltage (V), frequency (f), and/or period (T) of a drive signal or signals generated by the ultrasonic generator module 1008 and/or electrosurgery/RF generator module 1010.

The generator 1002 also may comprise an output device 1047 (FIG. 1), such as an output indicator, located, for example, on a front panel of the generator 1002 console. The output device 1047 includes one or more devices for providing a sensory feedback to a user. Such devices may comprise, for example, visual feedback devices (e.g., a visual feedback device may comprise incandescent lamps, light emitting diodes (LEDs), graphical user interface, display, analog indicator, digital indicator, bar graph display, digital alphanumeric display, liquid crystal display (LCD) screen, LED indicators), audio feedback devices (e.g., an audio feedback device may comprise speaker, buzzer, audible, computer generated tone, computerized speech, voice user interface (VUI) to interact with computers through a voice/speech platform), or tactile feedback devices (e.g., a tactile feedback device comprises any type of vibratory feedback, haptic actuator).

Although certain modules and/or blocks of the generator 1002 may be described by way of example, it can be appreciated that a greater or lesser number of modules and/or blocks may be used and still fall within the scope of the forms. Further, although various forms may be described in terms of modules and/or blocks to facilitate description, such modules and/or blocks may be implemented by one or more hardware components, e.g., processor, Digital Signal Processor (DSP), Programmable Logic Devices (PLD), Complex Programmable Logic Device (CPLD), Field Programmable Gate Array (FPGA), Application Specific Integrated Circuit (ASIC), circuit, register and/or software component, e.g., program, subroutine, logic and/or combinations of hardware and software components. Also, in some forms, the various modules described herein may be implemented utilizing similar hardware positioned within the surgical instruments 1004, 1006, 1202 (i.e., the generator 1002 may be omitted).

In one form, the ultrasonic generator module 1008 and electrosurgery/RF drive module 1010 may comprise one or more embedded applications implemented as firmware, software, hardware, or any combination thereof. The modules 1008, 1010 may comprise various executable modules such as software, programs, data, drivers, application program interfaces (APIs), and so forth. The firmware may be stored in any data storage component such as, for example, NVM, such as in bit-masked read-only memory (ROM) or flash memory. In various implementations, storing the firmware in ROM may preserve flash memory. The NVM may comprise other types of memory including, for example, programmable ROM (PROM), erasable programmable ROM (EPROM), EEPROM, or battery backed random-access memory (RAM) such as dynamic RAM (DRAM), Double-Data-Rate DRAM (DDRAM), and/or synchronous DRAM (SDRAM).

In one form, the modules 1008, 1010 comprise a hardware component implemented as a processor for executing program instructions for monitoring various measurable characteristics of the surgical instruments 1004, 1006, 1202 and generating a corresponding output control signals for operating the surgical instruments 1004, 1006, 1202. In forms in which the generator 1002 is used in conjunction with the ultrasonic surgical instrument 1004, the output control signal may drive the ultrasonic transducer 1014 in cutting and/or coagulation operating modes. Electrical characteristics of the ultrasonic surgical instrument 1004 and/or tissue may be measured and used to control operational aspects of the generator 1002 and/or provided as feedback to the user. In forms in which the generator 1002 is used in conjunction with the electrosurgical instrument 1006, the output control signal may supply electrical energy (e.g., RF energy) to the end effector 1032 in cutting, coagulation and/or desiccation modes. Electrical characteristics of the electrosurgical instrument 1006 and/or tissue may be measured and used to control operational aspects of the generator 1002 and/or provide feedback to the user. In various forms, as previously discussed, the hardware component may be implemented as a DSP, PLD, FPGA, ASIC, circuits, and/or registers. In one form, the processor may be configured to store and execute computer software program instructions to generate the step function output signals for driving various components of the surgical instruments 1004, 1006, 1202, such as the ultrasonic transducer 1014 and the end effectors 1026, 1032.

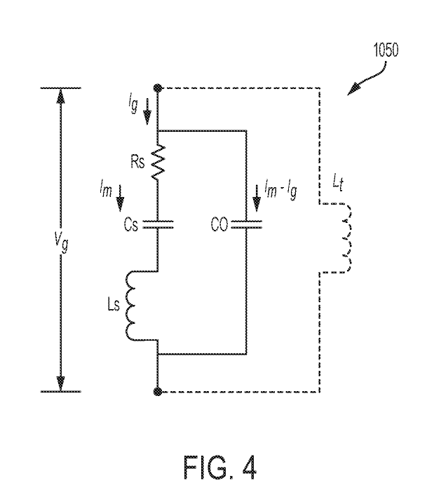

FIG. 4 illustrates an equivalent circuit 1050 of an ultrasonic transducer, such as the ultrasonic transducer 1014, according to one form. The equivalent circuit 1050 comprises a first “motional” branch having a serially connected inductance Lₛ, resistance Rₛ and capacitance Cₛ that define the electromechanical properties of the resonator, and a second capacitive branch having a static capacitance Cₒ. Drive current I_{g} may be received from a generator at a drive voltage V_{g}, with motional current Iₘ flowing through the first branch and current I_{g} -Iₘ flowing through the capacitive branch. Control of the electromechanical properties of the ultrasonic transducer may be achieved by suitably controlling I_{g} and V_{g}. As explained above, conventional generator architectures may include a tuning inductor Lₜ (shown in phantom in FIG. 4) for tuning out in a parallel resonance circuit the static capacitance Co at a resonant frequency so that substantially all of generator's current output I_{g} flows through the motional branch. In this way, control of the motional branch current Iₘ is achieved by controlling the generator current output I_{g}. The tuning inductor Lₜ is specific to the static capacitance Cₒ of an ultrasonic transducer, however, and a different ultrasonic transducer having a different static capacitance may call for a different tuning inductor Lₜ. Moreover, because the tuning inductor Lₜ is matched to the nominal value of the static capacitance Cₒ at a least one resonant frequency, accurate control of the motional branch current Iₘ is assured at that frequency, and as frequency shifts down with transducer temperature, accurate control of the motional branch current is compromised.

Forms of the generator 1002 do not rely on a tuning inductor Lₜ to monitor the motional branch current Iₘ. Instead, the generator 1002 may use the measured value of the static capacitance Cₒ in between applications of power for a specific ultrasonic surgical instrument 1004 (along with drive signal voltage and current feedback data) to determine values of the motional branch current Iₘ on a dynamic and ongoing basis (e.g., in real-time). Such forms of the generator 1002 are therefore able to provide virtual tuning to simulate a system that is tuned or resonant with any value of static capacitance Cₒ at any frequency, and not just at least one resonant frequency dictated by a nominal value of the static capacitance Cₒ.

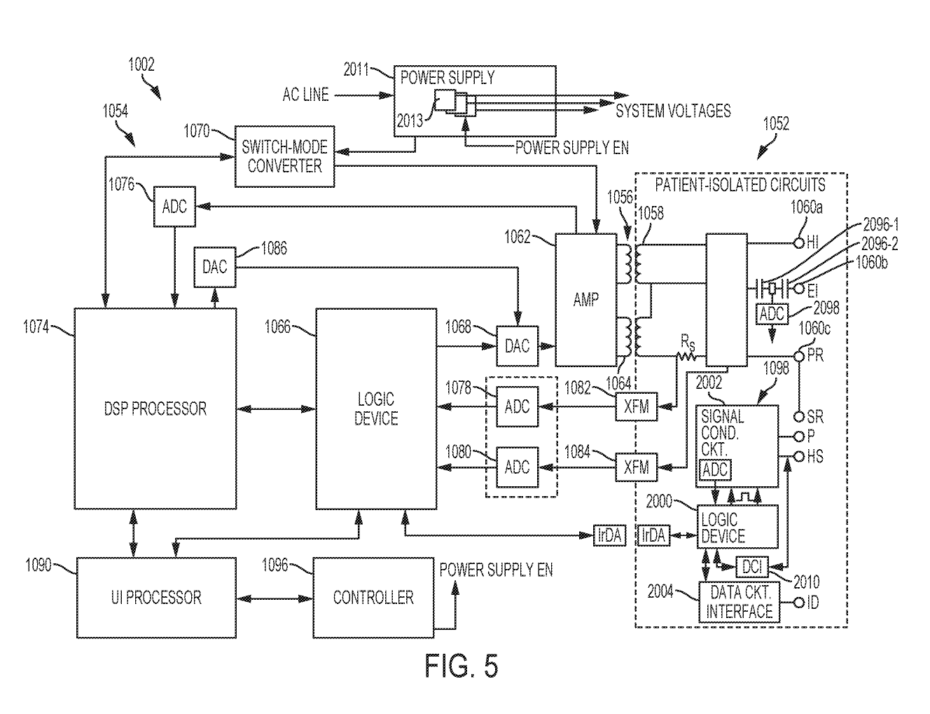

FIG. 5 is a simplified block diagram of one form of the generator 1002 for proving inductorless tuning as described above, among other benefits. Additional details of the generator 1002 are described in commonly assigned and contemporaneously filed U.S. patent application Ser. No. 12/896,360, titled SURGICAL GENERATOR FOR ULTRASONIC AND ELECTROSURGICAL DEVICES, now U.S. Pat. No. 9,060,775, the disclosure of which is incorporated herein by reference in its entirety. With reference to FIG. 5, the generator 1002 may comprise a patient isolated stage 1052 in communication with a non-isolated stage 1054 via a power transformer 1056. A secondary winding 1058 of the power transformer 1056 is contained in the patient isolated stage 1052 and may comprise a tapped configuration (e.g., a center-tapped or a non-center-tapped configuration) to define drive signal outputs 1060a, 1060b, 1060c for outputting drive signals to different surgical devices, such as, for example, an ultrasonic surgical instrument 1004 and an electrosurgical instrument 1006. In particular, drive signal outputs 1060a, 1060c may output an ultrasonic drive signal (e.g., a 420V root mean square [RMS] drive signal) to an ultrasonic surgical instrument 1004, and drive signal outputs 1060b, 1060c may output an electrosurgical drive signal (e.g., a 100V RMS drive signal) to an electrosurgical instrument 1006, with drive signal output 1060b corresponding to the center tap of the power transformer 1056.

In certain forms, the ultrasonic and electrosurgical drive signals may be provided simultaneously to distinct surgical instruments and/or to at least one surgical instrument having the capability to deliver ultrasonic and electrosurgical energy to tissue, such as multifunction device 1202 (FIGS. 1 and 3). It will be appreciated that the electrosurgical signal, provided either to a dedicated electrosurgical instrument and/or to a combined multifunction ultrasonic/electrosurgical instrument may be either a therapeutic or sub-therapeutic level signal. For example, the ultrasonic and RF signals can be delivered separately or simultaneously from a generator with at least one output port in order to provide the output signal to the surgical instrument, as will be discussed in more detail below. Accordingly, the generator can combine the ultrasonic and electrosurgical RF energies and deliver the combined energies to the multifunction ultrasonic/electrosurgical instrument. Bipolar electrodes can be placed on one or both jaws of the end effector. One jaw may be driven by ultrasonic energy in addition to electrosurgical RF energy, working simultaneously. The ultrasonic energy may be employed to dissect tissue while the electrosurgical RF energy may be employed for vessel sealing.

The non-isolated stage 1054 may comprise a power amplifier 1062 having an output connected to a primary winding 1064 of the power transformer 1056. In certain forms the power amplifier 1062 may be comprise a push-pull amplifier. For example, the non-isolated stage 1054 may further comprise a logic device 1066 for supplying a digital output to a digital-to-analog converter (DAC) 1068, which in turn supplies a corresponding analog signal to an input of the power amplifier 1062. In certain forms the logic device 1066 may comprise a PGA, FPGA, PLD, among other logic circuits, for example. The logic device 1066, by virtue of controlling the input of the power amplifier 1062 via the DAC 1068, may therefore control any of a number of parameters (e.g., frequency, waveform shape, waveform amplitude) of drive signals appearing at the drive signal outputs 1060a, 1060b, 1060c. In certain forms and as discussed below, the logic device 1066, in conjunction with a processor (e.g., a digital signal processor discussed below), may implement a number of digital signal processing (DSP)-based and/or other control algorithms to control parameters of the drive signals output by the generator 1002.

Power may be supplied to a power rail of the power amplifier 1062 by a switch-mode regulator 1070. In certain forms the switch-mode regulator 1070 may comprise an adjustable buck regulator, for example. The non-isolated stage 1054 may further comprise a first processor 1074, which in one form may comprise a DSP processor, for example, although in various forms any suitable processor may be employed. In certain forms the processor 1074 may control operation of the switch-mode regulator 1070 responsive to voltage feedback data received from the power amplifier 1062 by the processor 1074 via an Analog-to-Digital Converter 1076 (ADC). In one form, for example, the processor 1074 may receive as input, via the ADC 1076, the waveform envelope of a signal (e.g., an RF signal) being amplified by the power amplifier 1062. The processor 1074 may then control the switch-mode regulator 1070 (e.g., via a pulse-width modulated (PWM) output) such that the rail voltage supplied to the power amplifier 1062 tracks the waveform envelope of the amplified signal. By dynamically modulating the rail voltage of the power amplifier 1062 based on the waveform envelope, the efficiency of the power amplifier 1062 may be significantly improved relative to a fixed rail voltage amplifier schemes.

In certain forms, the logic device 1066, in conjunction with the processor 1074, may implement a direct digital synthesizer (DDS) control scheme to control the waveform shape, frequency and/or amplitude of drive signals output by the generator 1002. In one form, for example, the logic device 1066 may implement a DDS control algorithm by recalling waveform samples stored in a dynamically-updated look-up table (LUT), such as a RAM LUT, which may be embedded in an FPGA. This control algorithm is particularly useful for ultrasonic applications in which an ultrasonic transducer, such as the ultrasonic transducer 1014, may be driven by a clean sinusoidal current at its resonant frequency. Because other frequencies may excite parasitic resonances, minimizing or reducing the total distortion of the motional branch current may correspondingly minimize or reduce undesirable resonance effects. Because the waveform shape of a drive signal output by the generator 1002 is impacted by various sources of distortion present in the output drive circuit (e.g., the power transformer 1056, the power amplifier 1062), voltage and current feedback data based on the drive signal may be input into an algorithm, such as an error control algorithm implemented by the processor 1074, which compensates for distortion by suitably pre-distorting or modifying the waveform samples stored in the LUT on a dynamic, ongoing basis (e.g., in real-time). In one form, the amount or degree of pre-distortion applied to the LUT samples may be based on the error between a computed motional branch current and a current waveform shape, with the error being determined on a sample-by-sample basis. In this way, the pre-distorted LUT samples, when processed through the drive circuit, may result in a motional branch drive signal having the waveform shape (e.g., sinusoidal) for optimally driving the ultrasonic transducer. In such forms, the LUT waveform samples will therefore not represent the waveform shape of the drive signal, but rather the waveform shape to ultimately produce the waveform shape of the motional branch drive signal when distortion effects are taken into account.

The non-isolated stage 1054 may further comprise an ADC 1078 and an ADC 1080 coupled to the output of the power transformer 1056 via respective isolation transformers 1082, 1084 for respectively sampling the voltage and current of drive signals output by the generator 1002. In certain forms, the ADCs 1078, 1080 may be configured to sample at high speeds (e.g., 80 million samples per seconds [MSPS]) to enable oversampling of the drive signals. In one form, for example, the sampling speed of the ADCs 1078, 1080 may enable approximately 200×(depending on frequency) oversampling of the drive signals. In certain forms, the sampling operations of the ADC 1078, 1080 may be performed by a singe ADC receiving input voltage and current signals via a two-way multiplexer. The use of high-speed sampling in forms of the generator 1002 may enable, among other things, calculation of the complex current flowing through the motional branch (which may be used in certain forms to implement DDS-based waveform shape control described above), accurate digital filtering of the sampled signals, and calculation of real power consumption with a high degree of precision. Voltage and current feedback data output by the ADCs 1078, 1080 may be received and processed (e.g., first-in first-out [FIFO] buffering, multiplexing) by the logic device 1066 and stored in data memory for subsequent retrieval by, for example, the processor 1074. As noted above, voltage and current feedback data may be used as input to an algorithm for pre-distorting or modifying LUT waveform samples on a dynamic and ongoing basis. In certain forms, the stored voltage and current feedback data pair may be indexed based on, or otherwise associated with, a corresponding LUT sample that was output by the logic device 1066 when the voltage and current feedback data pair was acquired. Synchronization of the LUT samples and the voltage and current feedback data in this manner contributes to the correct timing and stability of the pre-distortion algorithm.

In certain forms, the voltage and current feedback data may be used to control the frequency and/or amplitude (e.g., current amplitude) of the drive signals. In one form, for example, voltage and current feedback data may be used to determine impedance phase. The frequency of the drive signal may then be controlled to minimize or reduce the difference between the determined impedance phase and an impedance phase set point (e.g.,0°), thereby minimizing or reducing the effects of harmonic distortion and correspondingly enhancing impedance phase measurement accuracy. The determination of phase impedance and a frequency control signal may be implemented in the processor 1074, for example, with the frequency control signal being supplied as input to a DDS control algorithm implemented by the logic device 1066.

In another form, for example, the current feedback data may be monitored in order to maintain the current amplitude of the drive signal at a current amplitude set point. The current amplitude set point may be specified directly or determined indirectly based on specified voltage amplitude and power set points. In certain forms, control of the current amplitude may be implemented by control algorithm, such as, for example, a proportional-integral-derivative (PID) control algorithm, in the processor 1074. Variables controlled by the control algorithm to suitably control the current amplitude of the drive signal may include, for example, the scaling of the LUT waveform samples stored in the logic device 1066 and/or the full-scale output voltage of the DAC 1068 (which supplies the input to the power amplifier 1062) via a DAC 1086.

The non-isolated stage 1054 may further comprise a second processor 1090 for providing, among other things user interface (UI) functionality. Examples of UI functionality supported by the UI processor 1090 may include audible and visual user feedback, communication with peripheral devices (e.g., via a Universal Serial Bus (USB) interface), communication with the foot switch 1020, communication with an input device 1009 (e.g., a touch screen display) and communication with an output device 1047 (e.g., a speaker). The UI processor 1090 may communicate with the processor 1074 and the logic device 1066 (e.g., via serial peripheral interface (SPI) buses). Although the UI processor 1090 may primarily support UI functionality, it also may coordinate with the processor 1074 to implement hazard mitigation in certain forms. For example, the UI processor 1090 may be programmed to monitor various aspects of user input and/or other inputs (e.g., touch screen inputs, foot switch 1020 inputs (FIG. 3), temperature sensor inputs) and may disable the drive output of the generator 1002 when an erroneous condition is detected.

In certain forms, the processor 1074 and the UI processor 1090, for example, may determine and monitor the operating state of the generator 1002. For the processor 1074, the operating state of the generator 1002 may dictate, for example, which control and/or diagnostic processes are implemented by the processor 1074. For the UI processor 1090, the operating state of the generator 1002 may dictate, for example, which elements of a user interface (e.g., display screens, sounds) are presented to a user. The respective DSP and UI processors 1074, 1090 may independently maintain the current operating state of the generator 1002 and recognize and evaluate possible transitions out of the current operating state. The processor 1074 may function as the master in this relationship and determine when transitions between operating states are to occur. The UI processor 1090 may be aware of valid transitions between operating states and may confirm if a particular transition is appropriate. For example, when the processor 1074 instructs the UI processor 1090 to transition to a specific state, the UI processor 1090 may verify that requested transition is valid. In the event that a requested transition between states is determined to be invalid by the UI processor 1090, the UI processor 1090 may cause the generator 1002 to enter a failure mode.

The non-isolated stage 1054 may further comprise a controller 1096 for monitoring the input device 1045 (e.g., a capacitive touch sensor used for turning the generator 1002 on and off, a capacitive touch screen). In certain forms, the controller 1096 may comprise at least one processor and/or other controller device in communication with the UI processor 1090. In one form, for example, the controller 1096 may comprise a processor configured to monitor user input provided via one or more capacitive touch sensors. In one form, the controller 1096 may comprise a touch screen controller to control and manage the acquisition of touch data from a capacitive touch screen.

In certain forms, when the generator 1002 is in a “power off” state, the controller 1096 may continue to receive operating power (e.g., via a line from a power supply of the generator 1002, such as the power supply 2011 discussed below). In this way, the controller 1096 may continue to monitor an input device 1045 (e.g., a capacitive touch sensor located on a front panel of the generator 1002) for turning the generator 1002 on and off. When the generator 1002 is in the power off state, the controller 1096 may wake the power supply (e.g., enable operation of one or more DC/DC voltage converters 2013 of the power supply 2011) if activation of the “on/off” input device 1045 by a user is detected. The controller 1096 may therefore initiate a sequence for transitioning the generator 1002 to a “power on” state. Conversely, the controller 1096 may initiate a sequence for transitioning the generator 1002 to the power off state if activation of the “on/off” input device 1045 is detected when the generator 1002 is in the power on state. In certain forms, for example, the controller 1096 may report activation of the “on/off” input device 1045 to the UI processor 1090, which in turn implements the necessary process sequence for transitioning the generator 1002 to the power off state. In such forms, the controller 1096 may have no independent ability for causing the removal of power from the generator 1002 after its power on state has been established.

In certain forms, the controller 1096 may cause the generator 1002 to provide audible or other sensory feedback for alerting the user that a power on or power off sequence has been initiated. Such an alert may be provided at the beginning of a power on or power off sequence and prior to the commencement of other processes associated with the sequence.

In certain forms, the patient isolated stage 1052 may comprise an instrument interface circuit 1098 to, for example, provide a communication interface between a control circuit of a surgical device (e.g., a control circuit comprising hand piece switches) and components of the non-isolated stage 1054, such as, for example, the logic device 1066, the processor 1074 and/or the UI processor 1090. In one aspect, the logic device 1066 may be programmable. The instrument interface circuit 1098 may exchange information with components of the non-isolated stage 1054 via a communication link that maintains a suitable degree of electrical isolation between the patient isolated stages 1052, 1054, such as, for example, an infrared (IR)-based communication link. Power may be supplied to the instrument interface circuit 1098 using, for example, a low-dropout voltage regulator powered by an isolation transformer driven from the non-isolated stage 1054.

In one form, the instrument interface circuit 1098 may comprise a logic circuit 2000 (e.g., logic circuit, programmable logic circuit, PGA, FPGA, PLD, CPLD, ASIC) in communication with a signal conditioning circuit 2002. The signal conditioning circuit 2002 may be configured to receive a periodic signal from the logic circuit 2000 (e.g., a 2 kHz square wave) to generate a bipolar interrogation signal having an identical frequency. The interrogation signal may be generated, for example, using a bipolar current source fed by a differential amplifier. The interrogation signal may be communicated to a surgical device control circuit (e.g., by using a conductive pair in a cable that connects the generator 1002 to the surgical device) and monitored to determine a state or configuration of the control circuit. The control circuit may comprise a number of switches, resistors and/or diodes to modify one or more characteristics (e.g., amplitude, rectification) of the interrogation signal such that a state or configuration of the control circuit is discernable based on the one or more characteristics. In one form, for example, the signal conditioning circuit 2002 may comprise an ADC for generating samples of a voltage signal appearing across inputs of the control circuit resulting from passage of interrogation signal therethrough. The logic circuit 2000 (or a component of the non-isolated stage 1054) may then determine the state or configuration of the control circuit based on the ADC samples.

In one form, the instrument interface circuit 1098 may comprise a first data circuit interface 2004 to enable information exchange between the logic circuit 2000 (or other element of the instrument interface circuit 1098) and a first data circuit disposed in or otherwise associated with a surgical device. In certain forms, for example, a first data circuit 2006 (FIG. 2) may be disposed in a cable integrally attached to a surgical device hand piece, or in an adaptor for interfacing a specific surgical device type or model with the generator 1002. The data circuit 2006 may be implemented in any suitable manner and may communicate with the generator according to any suitable protocol including, for example, as described herein with respect to the data circuit 6006. In certain forms, the first data circuit may comprise a NVM storage device, such as an EEPROM device, for example. In certain forms and referring again to FIG. 5, the first data circuit interface 2004 may be implemented separately from the logic circuit 2000 and comprise suitable circuit (e.g., discrete logic devices, a processor) to enable communication between the programmable logic circuit 2000 and the first data circuit. In other forms, the first data circuit interface 2004 may be integral with the logic circuit 2000.

In certain forms, the first data circuit 2006 may store information pertaining to the particular surgical device with which it is associated. Such information may include, for example, a model number, a serial number, a number of operations in which the surgical device has been used, and/or any other type of information. This information may be read by the instrument interface circuit 1098 (e.g., by the logic circuit 2000), transferred to a component of the non-isolated stage 1054 (e.g., to logic device 1066, processor 1074 and/or UI processor 1090) for presentation to a user via an output device 1047 and/or for controlling a function or operation of the generator 1002. Additionally, any type of information may be communicated to first data circuit 2006 for storage therein via the first data circuit interface 2004 (e.g., using the logic circuit 2000). Such information may comprise, for example, an updated number of operations in which the surgical device has been used and/or dates and/or times of its usage.

As discussed previously, a surgical instrument may be detachable from a hand piece (e.g., surgical instrument 1024 may be detachable from hand piece 1004) to promote instrument interchangeability and/or disposability. In such cases, conventional generators may be limited in their ability to recognize particular instrument configurations being used and to optimize control and diagnostic processes accordingly. The addition of readable data circuits to surgical device instruments to address this issue is problematic from a compatibility standpoint, however. For example, designing a surgical device to remain backwardly compatible with generators that lack the requisite data reading functionality may be impractical due to, for example, differing signal schemes, design complexity, and cost. Forms of instruments discussed herein address these concerns by using data circuits that may be implemented in existing surgical instruments economically and with minimal design changes to preserve compatibility of the surgical devices with current generator platforms.

Additionally, forms of the generator 1002 may enable communication with instrument-based data circuits. For example, the generator 1002 may be configured to communicate with a second data circuit 2007 contained in an instrument (e.g., surgical instrument 1024) of a surgical device (FIG. 2). In some forms, the second data circuit 2007 may be implemented in a many similar to that of the data circuit 6006 described herein. The instrument interface circuit 1098 may comprise a second data circuit interface 2010 to enable this communication. In one form, the second data circuit interface 2010 may comprise a tri-state digital interface, although other interfaces also may be used. In certain forms, the second data circuit may generally be any circuit for transmitting and/or receiving data. In one form, for example, the second data circuit may store information pertaining to the particular surgical instrument with which it is associated. Such information may include, for example, a model number, a serial number, a number of operations in which the surgical instrument has been used, and/or any other type of information. In some forms, the second data circuit 2007 may store information about the electrical and/or ultrasonic properties of an associated ultrasonic transducer 1014, end effector 1026, or ultrasonic drive system. For example, the first data circuit 2006 may indicate a burn-in frequency slope, as described herein. Additionally or alternatively, any type of information may be communicated to second data circuit for storage therein via the second data circuit interface 2010 (e.g., using the logic circuit 2000). Such information may comprise, for example, an updated number of operations in which the instrument has been used and/or dates and/or times of its usage. In certain forms, the second data circuit may transmit data acquired by one or more sensors (e.g., an instrument-based temperature sensor). In certain forms, the second data circuit may receive data from the generator 1002 and provide an indication to a user (e.g., an LED indication or other visible indication) based on the received data.

In certain forms, the second data circuit and the second data circuit interface 2010 may be configured such that communication between the logic circuit 2000 and the second data circuit can be effected without providing additional conductors for this purpose (e.g., dedicated conductors of a cable connecting a hand piece to the generator 1002). In one form, for example, information may be communicated to and from the second data circuit using a serial bus communication scheme implemented on existing cabling, such as one of the conductors used transmit interrogation signals from the signal conditioning circuit 2002 to a control circuit in a hand piece. In this way, design changes or modifications to the surgical device that might otherwise be necessary are minimized or reduced. Moreover, because different types of communications implemented over a common physical channel can be frequency-band separated, the presence of a second data circuit may be “invisible” to generators that do not have the requisite data reading functionality, thus enabling backward compatibility of the surgical device instrument.

In certain forms, the patient isolated stage 1052 may comprise at least one blocking capacitor 2096-1 connected to the drive signal output 1060b to prevent passage of DC current to a patient. At least one blocking capacitor may be employed to comply with medical regulations or standards, for example. While failure in capacitor designs comprising at least one capacitor is relatively uncommon, such failure may nonetheless have negative consequences. In one form, a second blocking capacitor 2096-2 may be provided in series with the blocking capacitor 2096-1, with current leakage from a point between the blocking capacitors 2096-1, 2096-2 being monitored by, for example, an ADC 2098 for sampling a voltage induced by leakage current. The samples may be received by the logic circuit 2000, for example. Based changes in the leakage current (as indicated by the voltage samples in the form of FIG. 5), the generator 1002 may determine when at least one of the blocking capacitors 2096-1, 2096-2 has failed. Accordingly, the form of FIG. 5 provides a benefit over designs comprising at least one capacitor having a single point of failure.

In certain forms, the non-isolated stage 1054 may comprise a power supply 2011 for outputting DC power at a suitable voltage and current. The power supply may comprise, for example, a 400 W power supply for outputting a 48 VDC system voltage. The power supply 2011 may further comprise one or more DC/DC voltage converters 2013 for receiving the output of the power supply to generate DC outputs at the voltages and currents to various components of the generator 1002. As discussed above in connection with the controller 1096, one or more of the DC/DC voltage converters 2013 may receive an input from the controller 1096 when activation of the “on/off” input device 1045 by a user is detected by the controller 1096 to enable operation of, or wake, the DC/DC voltage converters 2013.

Having described operational details of various forms of the surgical system 10 (FIG. 1) operations for the surgical system 10 may be further described generally in terms of a process for cutting and coagulating tissue employing a surgical instrument comprising an input device 1045 and the generator 1002. Although a particular process is described in connection with the operational details, it can be appreciated that the process merely provides an example of how the general functionality described herein can be implemented by the surgical system 10. Further, the given process does not necessarily have to be executed in the order presented herein unless otherwise indicated. As previously discussed, the input device 1045 may be employed to program the output (e.g., impedance, current, voltage, frequency) of the surgical instruments 1004, 1006, 1202 (FIG. 1).

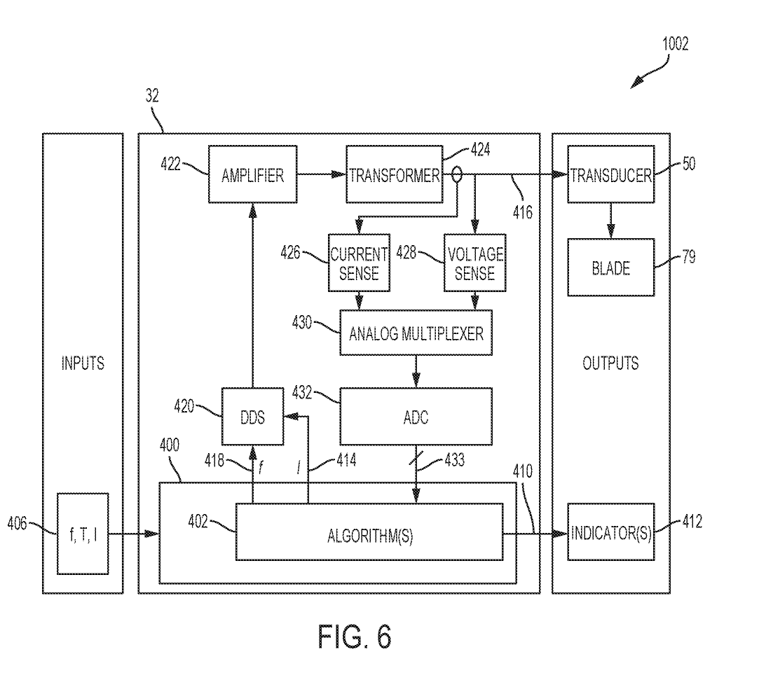

FIG. 6 illustrates one form of a drive system 32 of the generator 1002, which creates an ultrasonic electrical signal for driving an ultrasonic transducer, also referred to as a drive signal. The drive system 32 is flexible and can create an electrical output drive signal 416 at a frequency and power level setting for driving the ultrasonic transducer 50. In various forms, the generator 1002 may comprise several separate functional elements, such as modules and/or blocks. Although certain modules and/or blocks may be described by way of example, it can be appreciated that a greater or lesser number of modules and/or blocks may be used and still fall within the scope of the forms. Further, although various forms may be described in terms of modules and/or blocks to facilitate description, such modules and/or blocks may be implemented by one or more hardware components, e.g., processor, DSP, PLD, CPLD, FPGA, ASIC, circuit, register and/or software component, e.g., program, subroutine, logic and/or combinations of hardware and software components.

In one form, the generator 1002 drive system 32 may comprise one or more embedded applications implemented as firmware, software, hardware, or any combination thereof. The generator 1002 drive system 32 may comprise various executable modules such as software, programs, data, drivers, application program interfaces (APIs), and so forth. The firmware may be in NVM, such as in bit-masked ROM or flash memory. In various implementations, storing the firmware in ROM may preserve flash memory. The NVM may comprise other types of memory including, for example, PROM, EPROM, EEPROM, or battery backed RAM such as DRAM, DDRAM, and/or SDRAM.

In one form, the generator 1002 drive system 32 comprises a hardware component implemented as a processor 400 for executing program instructions for monitoring various measurable characteristics of the ultrasonic surgical instrument 1004 (FIG. 1) and generating a step function output signal for driving the ultrasonic transducer in cutting and/or coagulation operating modes. It will be appreciated by those skilled in the art that the generator 1002 and the drive system 32 may comprise additional or fewer components and a simplified version of the generator 1002 and the drive system 32 are described herein for conciseness and clarity. In various forms, as previously discussed, the hardware component may be implemented as a DSP, PLD, CPLD, FPGA, ASIC, circuit, and/or register. In one form, the processor 400 may be configured to store and execute computer software program instructions to generate the step function output signals for driving various components of the ultrasonic surgical instrument 1004, such as a transducer, an end effector, and/or a blade.

In one form, under control of one or more software program routines, the processor 400 executes the methods in accordance with the described forms to generate a step function formed by a stepwise waveform of drive signals comprising current (I), voltage (V), and/or frequency (f) for various time intervals or periods (T). The stepwise waveforms of the drive signals may be generated by forming a piecewise linear combination of constant functions over a plurality of time intervals created by stepping the generator 1002 drive signals, e.g., output drive current (I), voltage (V), and/or frequency (f). The time intervals or periods (T) may be predetermined (e.g., fixed and/or programmed by the user) or may be variable. Variable time intervals may be defined by setting the drive signal to a first value and maintaining the drive signal at that value until a change is detected in a monitored characteristic. Examples of monitored characteristics may comprise, for example, transducer impedance, tissue impedance, tissue heating, tissue transection, tissue coagulation, and the like. The ultrasonic drive signals generated by the generator 1002 include, without limitation, ultrasonic drive signals capable of exciting the ultrasonic transducer 50 in various vibratory modes such as, for example, the primary longitudinal mode and harmonics thereof as well flexural and torsional vibratory modes.

In one form, the executable modules comprise one or more algorithm(s) 402 stored in memory that when executed causes the processor 400 to generate a step function formed by a stepwise waveform of drive signals comprising current (I), voltage (V), and/or frequency (f) for various time intervals or periods (T). The stepwise waveforms of the drive signals may be generated by forming a piecewise linear combination of constant functions over two or more time intervals created by stepping the generator's 1002 output drive current (I), voltage (V), and/or frequency (f). The drive signals may be generated either for predetermined fixed time intervals or periods (T) of time or variable time intervals or periods of time in accordance with the one or more algorithm(s) 402. Under control of the processor 400, the generator 1002 steps (e.g., increment or decrement) the current (I), voltage (V), and/or frequency (f) up or down at a particular resolution for a predetermined period (T) or until a predetermined condition is detected, such as a change in a monitored characteristic (e.g., transducer impedance, tissue impedance). The steps can change in programmed increments or decrements. In other steps, the generator 1002 can increase or decrease the step adaptively based on measured system characteristics.

In operation, the user can program the operation of the generator 1002 using the input device 406 located on the front panel of the generator 1002 console. The input device 406 may comprise any suitable device that generates signals 408 that can be applied to the processor 400 to control the operation of the generator 1002. In various forms, the input device 406 includes buttons, switches, thumbwheels, keyboard, keypad, touch screen monitor, pointing device, remote connection to a general purpose or dedicated computer. In other forms, the input device 406 may comprise a suitable user interface. Accordingly, by way of the input device 406, the user can set or program the current (I), voltage (V), frequency (f), and/or period (T) for programming the step function output of the generator 1002. The processor 400 then displays the selected power level by sending a signal on line 410 to an output indicator 412.

In various forms, the output indicator 412 may provide visual, audible, and/or tactile feedback to the surgeon to indicate the status of a surgical procedure, such as, for example, when tissue cutting and coagulating is complete based on a measured characteristic of the ultrasonic surgical instrument 1004, e.g., transducer impedance, tissue impedance, or other measurements as subsequently described. By way of example, and not limitation, visual feedback comprises any type of visual indication device including incandescent lamps or LEDs, graphical user interface, display, analog indicator, digital indicator, bar graph display, digital alphanumeric display. By way of example, and not limitation, audible feedback comprises any type of buzzer, computer generated tone, computerized speech, VUI to interact with computers through a voice/speech platform. By way of example, and not limitation, tactile feedback comprises any type of vibratory feedback provided through an instrument housing handle assembly.

In one form, the processor 400 may be configured or programmed to generate a digital current signal 414 and a digital frequency signal 418. These signals 414, 418 are applied to a direct digital synthesizer (DDS) circuit 420 to adjust the amplitude and the frequency (f) of the current electrical output drive signal 416 to the ultrasonic transducer. The output of the DDS circuit 420 is applied to an amplifier 422 whose output is applied to a transformer 424. The output of the transformer 424 is the electrical output drive signal 416 applied to the ultrasonic transducer, which is coupled to a blade by way of a waveguide.

In one form, the generator 1002 comprises one or more measurement modules or components that may be configured to monitor measurable characteristics of the ultrasonic surgical instrument 1004 (FIG. 1). In the illustrated form, the processor 400 may be employed to monitor and calculate system characteristics. As shown, the processor 400 measures the impedance Z of the transducer by monitoring the current supplied to the ultrasonic transducer 50 and the voltage applied to the transducer. In one form, a current sensing circuit 426 is employed to sense the current flowing through the transducer and a voltage sensing circuit 428 is employed to sense the output voltage applied to the transducer. These signals may be applied to the ADC 432 via an analog multiplexer 430 circuit or switching circuit arrangement. The analog multiplexer 430 routes the appropriate analog signal to the ADC 432 for conversion. In other forms, multiple ADCs 432 may be employed for measured characteristic instead of the analog multiplexer 430 circuit. The processor 400 receives the digitized output 433 of the ADC 432 and calculates the transducer impedance Z based on the measured values of current and voltage. The processor 400 adjusts the electrical output drive signal 416 such that it can generate a power versus load curve. In accordance with programmed algorithm(s) 402, the processor 400 can step the electrical output drive signal 416, e.g., the current or frequency, in any suitable increment or decrement in response to the transducer impedance Z.

Having described operational details of various forms of the surgical system 10, operations for the surgical system 10 may be further described in terms of a process for cutting and coagulating a blood vessel employing a surgical instrument comprising the input device 1045 and the transducer impedance measurement capabilities described with reference to FIG. 6. Although a particular process is described in connection with the operational details, it can be appreciated that the process merely provides an example of how the general functionality described herein can be implemented by the surgical system 10. Further, the given process does not necessarily have to be executed in the order presented herein unless otherwise indicated.

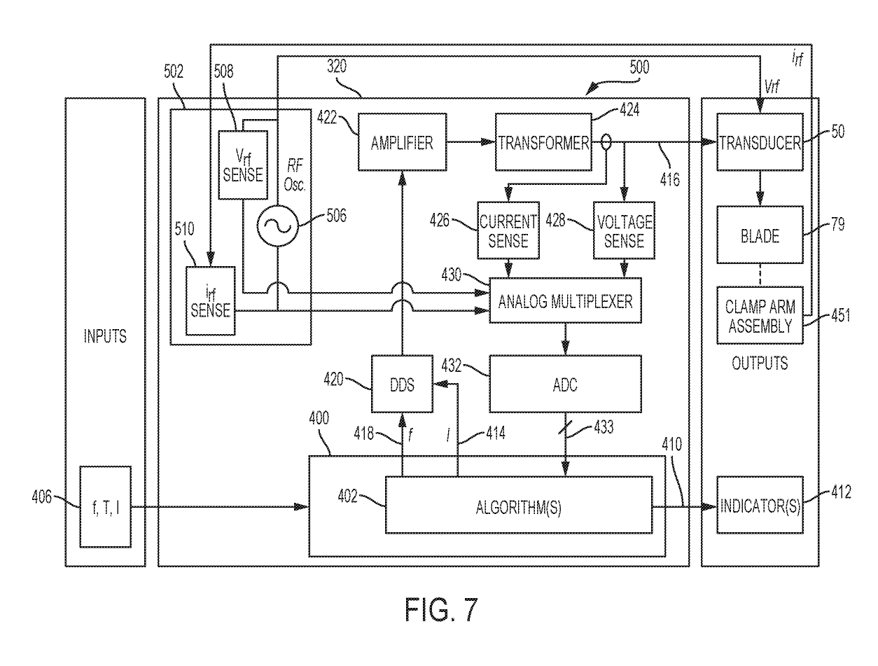

FIG. 7 illustrates one aspect of a drive system 320 of the generator 500 comprising the tissue impedance module 502. The drive system 320 generates the ultrasonic electrical output drive signal 416 to drive the ultrasonic transducer 50. In one aspect, the tissue impedance module 502 may be configured to measure the impedance Zt of tissue grasped between the blade 79 and the clamp arm assembly 451. The tissue impedance module 502 comprises an RF oscillator 506, a voltage sensing circuit 508, and a current sensing circuit 510. The voltage sensing circuit 508 and the current sensing circuit 510 respond to the RF voltage Vrf applied to the blade 79 electrode and the RF current irf flowing through the blade 79 electrode, the tissue, and the conductive portion of the clamp arm assembly 451. The sensed voltage Vrf and current Irf are converted to digital form by the ADC 432 via the analog multiplexer 430. The processor 400 receives the digitized output 433 of the ADC 432 and determines the tissue impedance Zt by calculating the ratio of the RF voltage Vrf to current Irf measured by the voltage sense circuit 508 and the current sensing circuit 510. In one aspect, the transection of the inner muscle layer and the tissue may be detected by sensing the tissue impedance Zt. Accordingly, detection of the tissue impedance Zt may be integrated with an automated process for separating the inner muscle layer from the outer adventitia layer prior to transecting the tissue without causing a significant amount of heating, which normally occurs at resonance.

In one form, the RF voltage Vrf applied to the blade 79 electrode and the RF current Irf flowing through the blade 79 electrode, the tissue, and the conductive portion of the clamp arm assembly 451 are suitable for vessel sealing and/or dissecting. Thus, the RF power output of the generator 500 can be selected for non-therapeutic functions such as tissue impedance measurements as well as therapeutic functions such as vessel sealing and/or dissection. It will be appreciated, that in the context of the present disclosure, the ultrasonic and the RF electrosurgical energies can be supplied by the generator either individually or simultaneously.

In various forms, feedback is provided by the output indicator 412 shown in FIGS. 6 and 7. The output indicator 412 is particularly useful in applications where the tissue being manipulated by the end effector is out of the user's field of view and the user cannot see when a change of state occurs in the tissue. The output indicator 412 communicates to the user that a change in tissue state has occurred. As previously discussed, the output indicator 412 may be configured to provide various types of feedback to the user including, without limitation, visual, audible, and/or tactile feedback to indicate to the user (e.g., surgeon, clinician) that the tissue has undergone a change of state or condition of the tissue. By way of example, and not limitation, as previously discussed, visual feedback comprises any type of visual indication device including incandescent lamps or LEDs, graphical user interface, display, analog indicator, digital indicator, bar graph display, digital alphanumeric display. By way of example, and not limitation, audible feedback comprises any type of buzzer, computer generated tone, computerized speech, VUI to interact with computers through a voice/speech platform. By way of example, and not limitation, tactile feedback comprises any type of vibratory feedback provided through the instrument housing handle assembly. The change of state of the tissue may be determined based on transducer and tissue impedance measurements as previously described, or based on voltage, current, and frequency measurements.

In one form, the various executable modules (e.g., algorithms) comprising computer readable instructions can be executed by the processor 400 (FIGS. 6, 7) portion of the generator 1002. In various forms, the operations described with respect to the algorithms may be implemented as one or more software components, e.g., program, subroutine, logic; one or more hardware components, e.g., processor, DSP, PLD, CPLD, FPGA, ASIC, circuit, register; and/or combinations of software and hardware. In one form, the executable instructions to perform the algorithms may be stored in memory. When executed, the instructions cause the processor 400 to determine a change in tissue state provide feedback to the user by way of the output indicator 412. In accordance with such executable instructions, the processor 400 monitors and evaluates the voltage, current, and/or frequency signal samples available from the generator 1002 and according to the evaluation of such signal samples determines whether a change in tissue state has occurred. As further described below, a change in tissue state may be determined based on the type of ultrasonic instrument and the power level that the instrument is energized at. In response to the feedback, the operational mode of the ultrasonic surgical instrument 1004 may be controlled by the user or may be automatically or semi-automatically controlled.

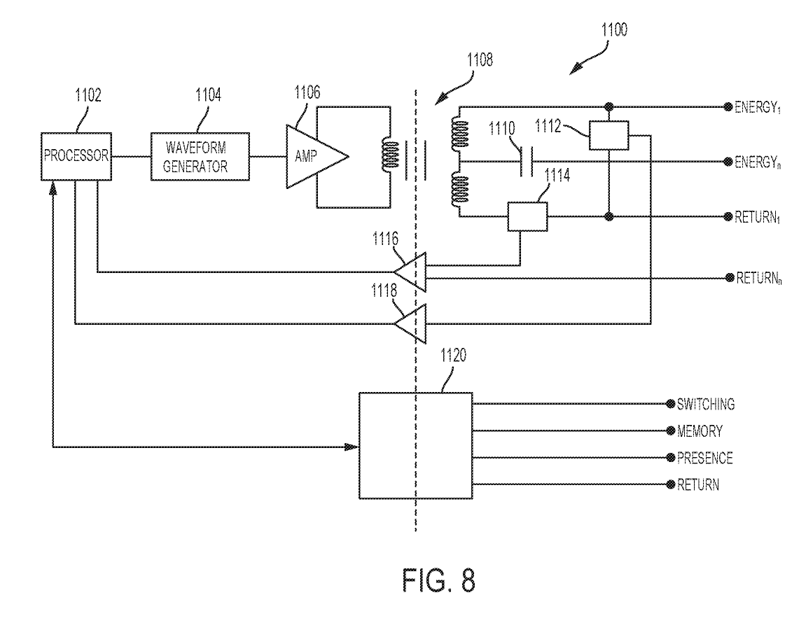

As noted above, at least one generator output can deliver multiple energy modalities (e.g., ultrasonic, bipolar or monopolar RF, irreversible and/or reversible electroporation, and/or microwave energy, among others) through a single port and these signals can be delivered separately or simultaneously to the end effector to treat tissue. FIG. 8 illustrates an example of a generator 1100 for delivering multiple energy modalities to a surgical instrument. The generator 1100 comprises a processor 1102 coupled to a waveform generator 1104. The processor 1102 and waveform generator 1104 are configured to generate a variety of signal waveforms based on information stored in a memory coupled to the processor 1102, not shown for clarity of disclosure. The digitally information associated with a waveform is provided to the waveform generator 1104 which includes one or more digital-to-analog (DAC) converters to convert the digital input into an analog output. The analog output is fed to an amplifier 1106 for signal conditioning and amplification. The conditioned and amplified output of the amplifier 1106 is coupled to a power transformer 1108. The signals are coupled across the power transformer 1108 to the secondary side, which is in the patient isolation side. A first signal of a first energy modality is provided to the surgical instrument between the terminals labeled ENERGY₁ and RETURN₁. A second signal of a second energy modality is coupled across a capacitor 1110 and is provided to the surgical instrument between the terminals labeled ENERGYₙ and RETURNₙ. The subscript n is used to indicate that up to n ENERGY/RETURN terminals may be provided, where n is a positive integer greater than 1. As an example, the first energy modality may be ultrasonic energy and the second energy modality may be RF energy. Nevertheless, in addition to ultrasonic and bipolar or monopolar RF energy modalities, other energy modalities include irreversible and/or reversible electroporation and/or microwave energy, among others. Also, although the example illustrated in FIG. 8 shows separate return paths RETURN₁ and RETURNₙ, it will be appreciated that at least one common return path may be provided for two or more energy modalities.

A voltage sensing circuit 1112 is coupled across the terminals labeled ENERGY₁ and RETURN₁ to measure the output voltage. A current sensing circuit 1114 is disposed in series with the RETURN₁ leg of the secondary side of the power transformer 1108 as shown to measure the output current. The outputs of the voltage sensing circuit 1112 is provided to an isolation transformer and ADC 1116 and the output of the current sensing circuit 1114 is provided to another isolation transformer and ADC 1118. The digital version of the output voltage and output current are fed back to the processor 1102. The output voltage and output current information can be employed to adjust the output voltage and current provided to the instrument and to compute output impedance, among other parameters. Input/output communications between the processor 1102 and patient isolated circuits is provided through an interface circuit 1120. It will be appreciated that a similar voltage sensing circuit may be provided across the ENERGYₙ and RETURNₙ terminals and a similar current sensing circuit may be disposed in series with the RETURNₙ leg.

As shown in FIG. 8, the generator 1100 comprising at least one output port can include a power transformer 1108 with a single output and with multiple taps to provide power in the form of one or more energy modalities, such as ultrasonic, bipolar or monopolar RF, irreversible and/or reversible electroporation, and/or microwave energy, among others, for example, to the end effector depending on the type of treatment of tissue being performed. For example, the generator 1100 can deliver energy with higher voltage and lower current to drive an ultrasonic transducer, with lower voltage and higher current to drive RF electrodes for sealing tissue, or with a coagulation waveform for spot coagulation using either monopolar or bipolar RF electrosurgical electrodes. The output waveform from the generator 1100 can be steered, switched, or filtered to provide the frequency to the end effector of the surgical instrument. The connection of a transducer to the generator 1100 output would be preferably located between the output labeled ENERGY1 and RETURN₁ as shown in FIG. 8. In one example, a connection of RF bipolar electrodes to the generator 1100 output would be preferably located between the output labeled ENERGYₙ and RETURNₙ. In the case of monopolar output, the preferred connections would be active electrode (e.g., pencil or other probe) to the ENERGYₙ output and a suitable return pad connected to the RETURNₙ output.

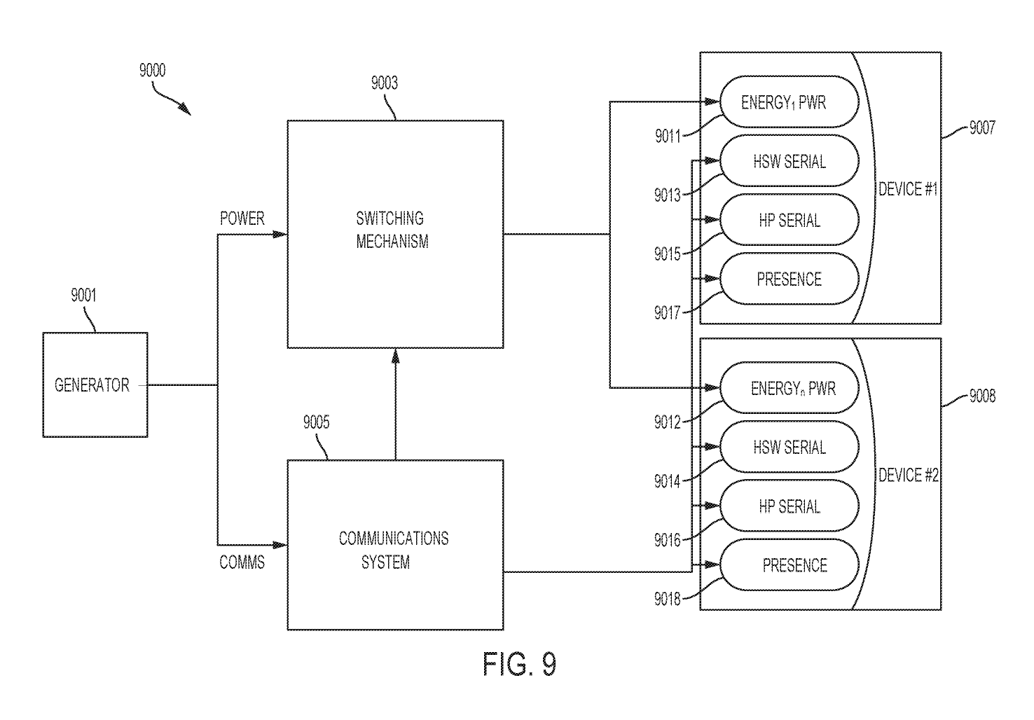

FIG. 9 shows a diagram of an electrosurgical system 9000 that allows for two ports on a generator 9001 and accounts for electrical isolation between two surgical instruments 9007, 9008. A scheme is provided for electrical isolation between the two instruments 9007, 9008 as they are located on the same patient isolation circuit. According to the configuration shown in FIG. 9, unintended electrical power feedback is prevented through the electrosurgical system 9000. In various aspects, one or more than one power field effect transistor (FET) or relays are used to electrically isolate the power lines for instruments 9007, 9008. According to one aspect, the power FETs or relays are controlled by a serial communication protocol.

As shown in FIG. 9, the generator 9001 is coupled to a power switching mechanism 9003 and a communications system 9005. In one aspect, the power switching mechanism 9003 comprises one or more than one power FET, such as a metal oxide semiconductor field effect transistor (MOSFET), and/or relays, such as electromechanical relays. In one aspect, the communications system 9005 comprises components for serial communication, microprocessor, ASIC/FPGA expansion, and time slicing functionalities. Time slicing can also apply to power signals. For example, when the instrument is operated at 330 kHz, a very short pulse can be transmitted at a different set of frequencies for charging. It also can be done by delivering power at the same frequency, but to the charging system rather than the tissue. This technique relies on certain relays or other switches to ensure that power cannot be delivered to the patient.

The power switching mechanism 9003 is coupled to the communications system 9005. The power switching mechanism 9003 and the communications system 9005 are coupled to surgical instruments 9007, 9009 (labeled device 1 and device 2). The surgical instruments 9007, 9009 comprise components for delivering multiple energy modalities to a plurality of surgical instruments where the multiple energy modalities include ultrasonic, bipolar or monopolar RF, reversible and/or irreversible electroporation, and/or microwave energy. As shown a first energy modality is provided at input terminal ENERGY₁ PWR 9011 of one surgical instrument 9007 and a another (or n) energy modality is provided at input terminal ENERGYₙ PWR 9012 of another surgical instrument 9008. Other inputs include handswitch (HSW) serial interfaces 9013, 9014, handpiece (HP) serial interfaces 9015, 9016, and presence interfaces 9017, 9018 of one surgical instrument 9007 and another surgical instrument 9008, respectively. A power switching mechanism 9003 is coupled to the first and second energy modalities inputs 9011, 9012 for the surgical instruments 9007, 9008. The communications system 9005 is coupled to the handswitch serial interface 9013, 9014, the handpiece serial interface 9015, 9016, and presence interface 9017, 9018 for the surgical instruments 9007, 9008. While two surgical instruments 9007, 9008 are shown in FIG. 9, there may be more than two devices according to other aspects of the present disclosure.

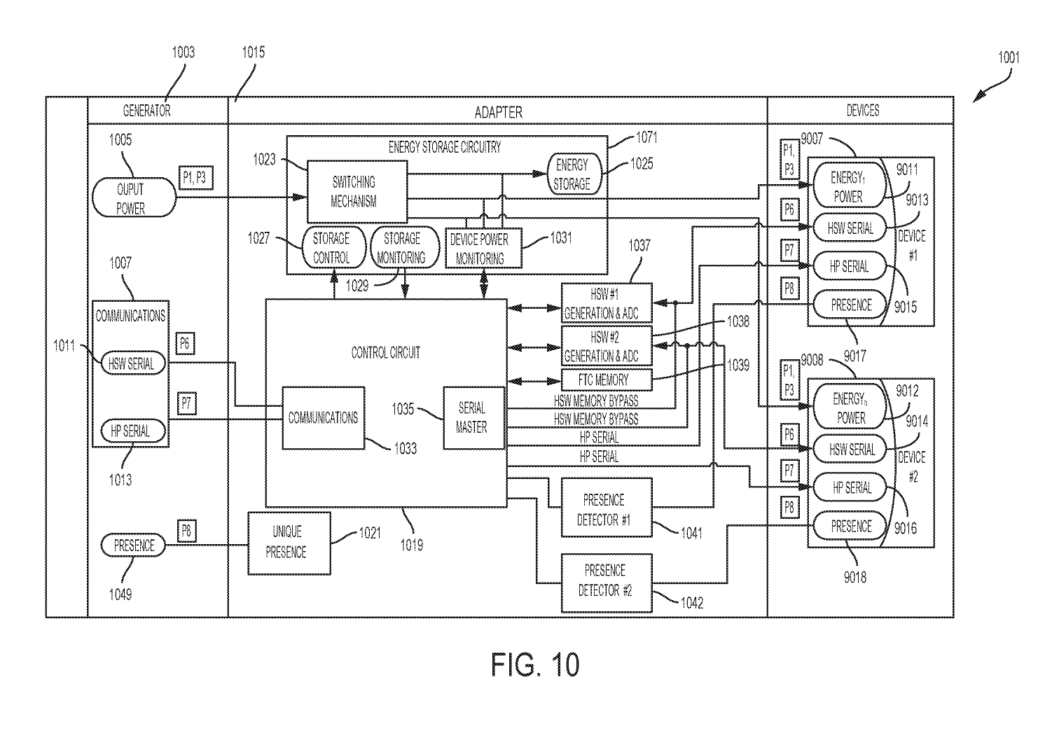

FIGS. 10-12 illustrate aspects of an interface with a generator to support two instruments simultaneously that allows the instruments to quickly switch between active/inactive by a user in a sterile field. FIGS. 10-12 describe multiple communication schemes which would allow for a super cap/battery charger and dual surgical instruments. The aspects of FIGS. 10-12 allow for communications to two surgical instruments in the surgical field from a generator with at least one communications port and allow for an operator in sterile field to switch between devices, for example, without modifying the surgical instruments.

FIG. 10 is a diagram of a communications architecture of system 1001 comprising a generator 1003 and surgical instruments 9007, 9008, which are shown in FIG. 9. According to FIG. 10, the generator 9001 is configured for delivering multiple energy modalities to a plurality of surgical instruments. As discussed herein the various energy modalities include, without limitation, ultrasonic, bipolar or monopolar RF, reversible and/or irreversible electroporation, and/or microwave energy modalities. The generator 9001 comprises a combined energy modality power output 1005, a communications interface 1007, and a presence interface 1049. According to the aspect of FIG. 10, the communications interface 1007 comprises an handswitch (HSW) serial interface 1011 and an handpiece (HP) serial interface 1013. The serial interfaces 1011, 1013 may comprise I²C, half duplex SPI, and/or Universal Asynchronous Receiver Transmitter (UART) components and/or functionalities. The generator 1003 provides the combined energy modalities power output 1005 to an adapter 1015, for example, a pass-through charger (PTC). The adapter 1015 comprises energy storage circuit 1071, control circuit 1019, a unique presence element 1021, and associated circuit discussed below. In one aspect, the presence element 1021 is a resistor. In another aspect, the presence element 1021 may be a bar code, Quick Response (QR) code, or similar code, or a value stored in memory such as, for example, a value stored in NVM. The presence element 1021 may be unique to the adapter 1015 so that, in the event that another adapter that did not use the same wire interfaces could not be used with the unique presence element 1021. In one aspect, the unique presence element 1021 is a resistor. The energy storage circuit 1071 comprises a switching mechanism 1023, energy storage device 1025, storage control 1027, storage monitoring component 1029, and a device power monitoring component 1031. The control circuit 1019 may comprise a processor, FPGA, PLD, CPLD, microcontroller, DSP, and/or ASIC, for example. According to the aspect shown in FIG. 10, an FPGA or microcontroller would act as an extension of an existing, similar computing hardware and allows for information to be relayed from on entity to another entity.

The switching mechanism 1023 is configured to receive the combined energy power output 1005 from the generator 1003 and it may be provided to the energy storage device 1025, surgical instrument 9007, and/or surgical instrument 9008. The device power monitoring component 1031 is coupled to the channels for the energy storage device 1025, surgical instrument 9007, surgical instrument 9008, and may monitor where power is flowing. The control circuit 1019 comprises communication interface 1033 coupled to the handswitch serial interface 1011 and an handpiece serial interface 1013 of the generator 1003. The control circuit 1019 is also coupled to the storage control 1027, storage monitoring component 1029, and device power monitoring component 1031 of the energy storage circuit 1071.

The control circuit 1019 further comprises a serial master interface 1035 that is coupled to handswitch (HSW) #1 circuit 1037 and handswitch (HSW) #2 circuit 1038, includes generation and ADC, a form of memory (non volatile or flash) 1039, along with a method for detecting the presence of an attached instrument (Presence) #1 circuit 1041 and Presence #2 circuit 1042, which includes a voltage or current source and ADC. The serial master interface 1035 also includes handswitch NVM bypass channels, which couple the serial master interface 1035 to the outputs of the handswitch #1 circuit 1037 and the handswitch #2 circuit 1038, respectively. The handswitch #1 circuit 1037 and handswitch #2 circuit 1038 are coupled to the handswitch serial interfaces 9013, 9014 of the surgical instruments 9007, 9008, respectively. The serial master interface 1035 further includes handpiece serial channels that are coupled to the handpiece serial interfaces 9015, 9016 of the surgical instruments 9007, 9008, respectively. Further, Presence #1 and Presence #2 circuits 1041, 1042 are coupled to the presence interfaces 9017, 9018 of the surgical instruments 9007, 9008, respectively.

The system 1001 allows the control circuit 1019, such as an FPGA, to communicate with more surgical devices using adapter 1015, which acts as an expansion adapter device. According to aspects, the adapter 1015 expands the Input/Output (I/O) capability of the generator 1003 control. The adapter 1015 may function as an extension of the central processing unit that allows commands to be transmitted over a bus between the adapter 1015 and the generator 1003 and unpacks the commands and use them to bit-bang over interfaces or to control connected analog circuit. The adapter 1015 also allows for reading in ADC values from connected surgical instruments 9007, 9008 and relay this information to the generator control and the generator control would then control the two surgical instruments 9007, 9008. According to aspects, the generator 1003 may control the surgical instruments 9007, 9008 as two separate state machines and may store the data.

Existing interfaces (the handswitch serial interface 1011 and the handpiece serial interface 1013 lines from generator 1003) may be used in a two-wire communication protocol that enables the generator 1003 control to communicate with multiple surgical devices connected to a dual port interface, similar to the topology of a universal serial bus (USB) hub. This allows interfacing with two separate surgical devices simultaneously. The system 1001 may be able to generate and read hand switch waveforms and be able to handle incoming handpiece serial buses. It would also monitor two separate presence elements in the surgical instruments 9007, 9008. In one aspect, the system 1001 may include a unique presence element and may have its own NVM.

Further, according to aspects, the control circuit 1019 may be controlled by the generator 1003. The communication between the adapter 1015 and connected surgical instruments 9007, 9008 may be relayed to generator control. The generator 1003 would control the waveform generation circuit connected to the adapter 1015 to simultaneously generate handswitch signals for surgical instruments 9007, 9008.

The system 1001 may allow surgical device activity that can be simultaneously detected/monitored for two surgical devices, even during activation. If upgradeable, the adapter 1015 would be capable of handling new surgical device communications protocols. Further, fast switching between surgical devices may be accomplished.

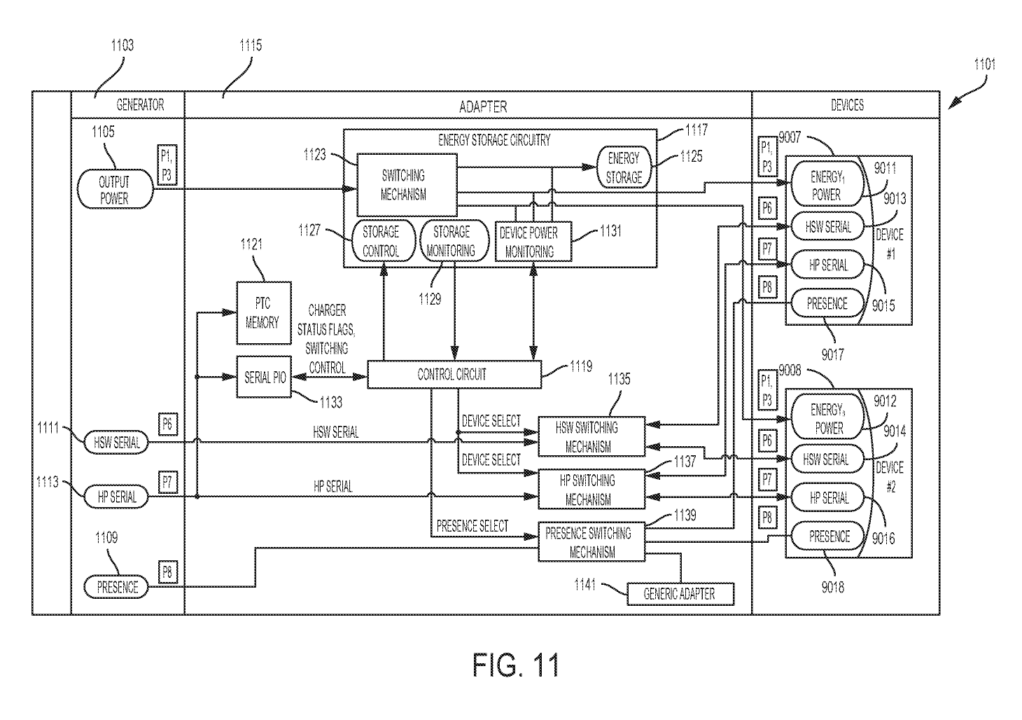

FIG. 11 illustrates a communication architecture of system 1101 of a generator 1103 and surgical instruments 9007, 9008 shown in FIG. 9. According to FIG. 11, the generator 1103 is configured for delivering multiple energy modalities to a plurality of surgical instruments. As discussed herein the various energy modalities include, without limitation, ultrasonic, bipolar or monopolar RF, reversible and/or irreversible electroporation, and/or microwave energy modalities. As shown in FIG. 11, the generator 1103 comprises a combined energy modality power output 1105, an handswitch (HSW) serial interface 1111, a handpiece (HP) serial interface 1113, and a presence interface 1109. The generator 1103 provides the power output 1105 to an adapter 1115. According to the aspect shown in FIG. 11, communications between the adapter 1115 and the generator 1103 may be done solely through serial interfaces, such as the handswitch serial and handpiece serial interfaces 1111, 1113. The generator 1103 may use these handswitch and handpiece serial interfaces 1111, 1113 to control which instrument the generator 1103 is communicating with. Further, switching between instruments could occur between handswitch frames or at a much slower rate.

The adapter 1115 comprises energy storage circuit 1117, control circuit 1119, an adapter memory 1121 (e.g., a NVM such as an EEPROM), a serial programmable input/output (PIO) integrated circuit 1133, an handswitch Switching Mechanism 1135, an handpiece Switching Mechanism 1137, a Presence Switching Mechanism 1139, and a Generic Adapter 1141. In one aspect, the serial PIO integrated circuit 1133 may be an addressable switch. The energy storage circuity 1117 comprises a switching mechanism 1123, energy storage device 1125, storage control component 1127, storage monitoring component 1129, and a device power monitoring component 1131. The control circuit 1119 may comprise a processor, FPGA, CPLD, PLD, microcontroller, DSP, and/or an ASIC, for example. According to the aspect of FIG. 11, an FPGA or microcontroller may have limited functionality and may solely comprise functionality for monitoring and communicating energy storage.

The switching mechanism 1123 is configured to receive the combined energy power output 1105 from the generator 1103 and it may be provided to the energy storage device 1125, surgical instrument 9007, and/or surgical instrument 9008. The device power monitoring component 1131 is coupled to the channels for the energy storage device 1125, surgical instrument 9007, surgical instrument 9008, and may monitor where power is flowing.

The control circuit 1119 is coupled to the serial PIO integrated circuit 1133 and the serial PIO integrated circuit 1133 is coupled to the handpiece serial interface 1113 of the generator 1103. The control circuit 1119 may receive information regarding charger status flags and switching controls from the serial PIO integrated circuit 1133. Further, the control circuit 1119 is coupled to the handswitch switching mechanism 1135, the handpiece switching mechanism 1137, and the presence switching mechanism 1139. According to the aspect of FIG. 11, the control circuit 1119 may be coupled to the handswitch (HSW) switching mechanism 1135 and the handpiece switching mechanism 1137 for device selection and the control circuit 1119 may be coupled to the presence switching Mechanism 1139 for presence selection.

The handswitch switching mechanism 1135, the handpiece switching mechanism 1137, and the presence switching mechanism 1139 are coupled to the handswitch serial interface 1111, the handpiece serial interface 1113, and the presence interface 1109 of generator 1103, respectively. Further, the handswitch switching mechanism 1135, the handpiece switching mechanism 1137, and the presence switching mechanism 1139 are coupled to the handswitch serial interfaces 9013, 9014, the handpiece serial interfaces 9015, 9016, and the presence interfaces 9017, 9018 of the surgical instruments 9007, 9008, respectively. Further, the presence switching mechanism 1139 is coupled to the generic adapter 1141.

The generator 1103 switches between monitoring the surgical instruments 9007, 9008. According to aspects, this switching may require the generator 1103 control to keep track of surgical instruments 9007, 9008 and run two separate state machines. The control circuit 1119 will need to remember which surgical instruments are connected, so that it can output an appropriate waveform to the ports where appropriate. The generator 1103 may generate/monitor hand switch signals, as well as communicating with serial NVM devices, such adapter memory 1121. The generator 1103 may maintain constant communication with the activating surgical instrument for the duration of the activation.

System 1101 also allows for a generic adapter presence element. When first plugged in or powered on, the adapter 1115 would present this adapter resistance to the generator 1103. The generator 1103 may then relay commands to the adapter 1115 to switch between the different presence elements corresponding to the different surgical instruments 9007, 9008 connected to it. Accordingly, the generator 1103 is able to use its existing presence resistance circuit. The NVM memory 1121 exists on the adapter 1115 for additional identification of the adapter and to provide a level of security. In addition, the adapter 1115 has a serial I/O device, i.e. serial PIO integrated circuit 1133. The serial PIO integrated circuit 1133 provides a communication link between the generator 1103 and the adapter 1115.

It may be possible to communicate over the handpiece serial bus using serial communications to handpiece NVMs and UART style communication to the control circuit 1119. According to one aspect, if SLOW serial communication is used(i.e. not overdrive) and a high speed serial protocol is used, system 1101 may need to ensure that the communications protocol does not generate a signal that looked like a serial reset pulse. This would allow better generator 1103 to adapter 1115 communications and faster switching times between surgical instruments 9007, 9008.

The system 1101 uses generator communications protocol and analog circuit and allows the generator to accomplish decision making. It is a simple and efficient solution that uses a small number of circuit devices.

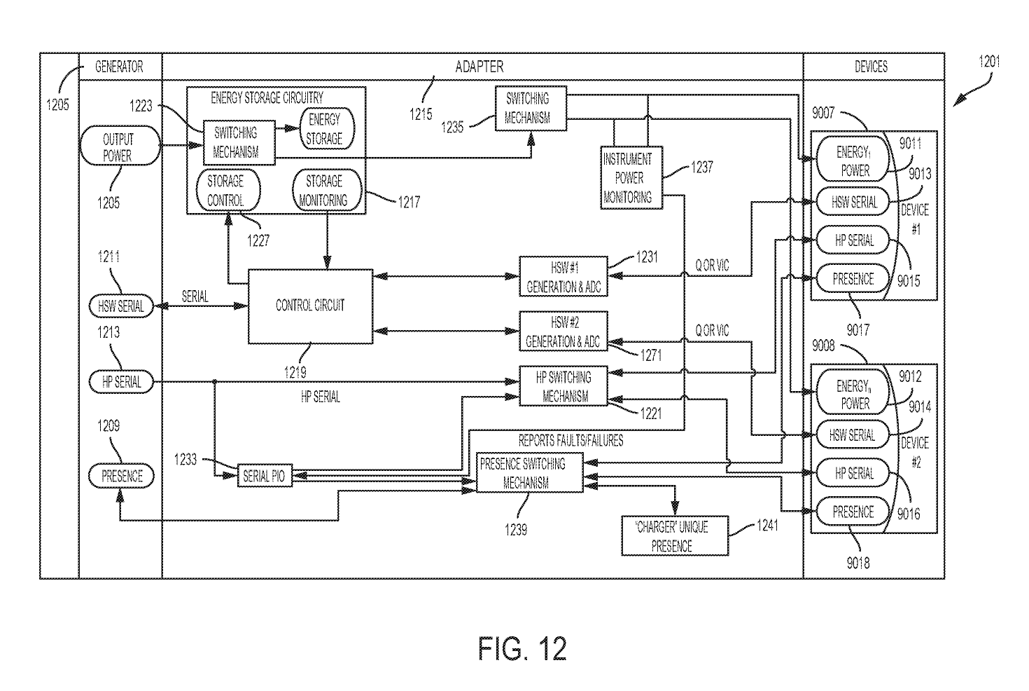

FIG. 12 illustrates a communications architecture of system 1201 of a generator 1203 and surgical instruments 9007, 9008 shown in FIG. 9. According to FIG. 12, the generator 1205 is configured for delivering multiple energy modalities to a plurality of surgical instruments. As discussed herein the various energy modalities include, without limitation, ultrasonic, bipolar or monopolar RF, reversible and/or irreversible electroporation, and/or microwave energy modalities. As shown in FIG. 12, the generator 1203 comprises a combined energy modality power output 1205, an handswitch serial interface 1211, an handpiece serial interface 1213, and a presence interface 1209. In one aspect, the handpiece serial interface 1213 allows for communication with the handpiece lines of the surgical instruments 9007, 9008 and also allows for control of the adapter 1215. The generator 1203 provides the combined energy modality power output 1205 to an adapter 1215. The adapter 1215 comprises energy storage circuit 1217, control circuit 1219, a serial PIO integrated circuit 1233 handswitch (HSW) #1 circuit 1231, handswitch (HSW) #2 circuit 1271, handpiece switching mechanism 1221, presence switching mechanism 1239 switching mechanism 1235, instrument power monitoring 1237, and unique presence 1241. As shown in FIG. 12, the handswitch #1 circuit 1231 and the handswitch #2 circuit 1271 may comprise generation and ADC circuits. In one aspect, handswitch #1 circuit 1231 and/or handswitch #2 circuit 1271 comprise generation circuit with the ability to generate handswitch waveforms.

The control circuit 1219 is coupled to the handswitch serial interface 1211 of the generator 1203 while the serial PIO integrated circuit 1233 is coupled to the handpiece serial interface 1213 as is the handpiece switching mechanism 1221. Further, the control circuit 1119 is coupled to the handswitch #1 circuit 1231 and the handswitch #2 circuit 1271. The control circuit 1119 may comprise a processor, FPGA, CPLD, PLD, microcontroller, and/or ASIC, for example. In the example shown in FIG. 12, the control circuit 1219 modulates two devices into at least one digital waveform, which enable the generator 1203 to perform the button monitoring and decision making. The control circuit 1219 also may allow for communication to two independent surgical instruments could receive either waveform. The serial PIO integrated circuit 1233 is further coupled to the handpiece switching mechanism 1221, the instrument power monitoring 1237, and the presence switching mechanism 1239. The instrument power monitoring 1237 and the serial PIO integrated circuit 1233 may communicate results and failures to the generator 1203.

The switching mechanism 1223 is configured to receive the combined RF/Ultrasonic power output 1205 from the generator 1203 and it may be provided to the energy storage device 1225 or the switching mechanism 1235. The control circuit 1219 is also coupled to the storage control 1227 and storage monitoring 1229 of the energy storage circuit 1217. The switching mechanism 1235 may provide the power output received from the switching mechanism 1223 to surgical instrument 9007, and/or surgical instrument 9008. The instrument power monitoring 1237 is coupled to the channels for the power output to the surgical instrument 9007 and surgical instrument 9008. The instrument power monitoring 1237 also may ensure that the switching mechanism 1235 is outputting power to correct location.

The handswitch #1 circuit 1231 and the handswitch #2 block 1271 are coupled to the handswitch serial interfaces 9013, 9014 of the surgical instruments 9007, 9008, respectively. The handpiece switching mechanism 1221 is coupled to the handpiece serial interface 1213 of the generator 1203 and to the handpiece serial interfaces 9015, 9016 of the surgical instruments 9007, 9008, respectively. Further, the presence switching mechanism 1239 is coupled to the presence interface 1209 of the generator 1203 and to the Presence Interfaces 9017, 9018 of the surgical instruments 9007, 9008, respectively. Further, Presence Switching mechanism is coupled to the unique presence 1241. In one aspect, different instrument presence elements may be switched on an on-demand basis using serial I/O or an adapter micro protocol.

A first communications protocol will be used to communicate to the control circuit 1219 on the adapter 1215. The generator 1205 also may have the ability to monitor surgical instruments 9007, 9008 at once. The adapter 1215 may comprise circuit to provide handswitch signal generation (e.g., in handswitch #1 circuit 1231 and handswitch #2 circuit 1271) along with ADCs to interpret this data. The adapter 1215 may modulate two surgical instrument signals into at least a first waveform and may have the ability to read in the first and second waveforms. In various aspects, the second waveforms may be interpreted and translated into the format of the first waveforms. Further, the first protocol has the ability to send 12 bits at 615 bits/sec.

The control circuit 1219 may take the handswitch data from surgical instruments 9007, 9008 and modulate it into a first protocol. There are a few ways of doing this, but it may mean that surgical devices 9007, 9008 may comprises a first protocol functionality. The system 1201 could communicate 4-6 buttons from the surgical instrument 9007 and 4-6 buttons from the surgical instrument 9008 in the first protocol frame. Alternatively, the system 1201 could use some form of addressing to access the surgical instruments 9007, 9008. The control circuit 1219 may have the ability to address separate devices by having the generator 1203 send the control circuit 1219 different addresses split into two different address spaces, one for surgical instrument 9007 and one for surgical instrument 9008.

The handpiece communications may involve some form of switch that could either be controlled via a serial I/O device or through the control circuit 1219 via a first protocol style communication interface from the generator 1203. In one aspect, energy storage monitoring 1229 and switching between surgical instruments 9007, 9008 and charging states could be handled in this manner as well. Certain first protocol addresses could be assigned to the data from the energy storage circuit 1225 and to the surgical instruments 9007, 9008 themselves. Presence elements could also be switched in with this format. Further, in one aspect, the control circuit 1219 may translate frames into a separate format, which may mean that the control circuit 1219 might need to make some decisions on whether button presses on surgical instruments 9007, 9008 are valid or not. The system 1201 would, however, allow the generator 1203 to fully monitor the surgical instruments 9007, 9008 at the same time time-slicing or handling a new communications protocol on the handswitch serial interface 1211 of the generator 1203. The system 1201 uses generator communications to simultaneously detect the activity of two surgical devices, even during activation.

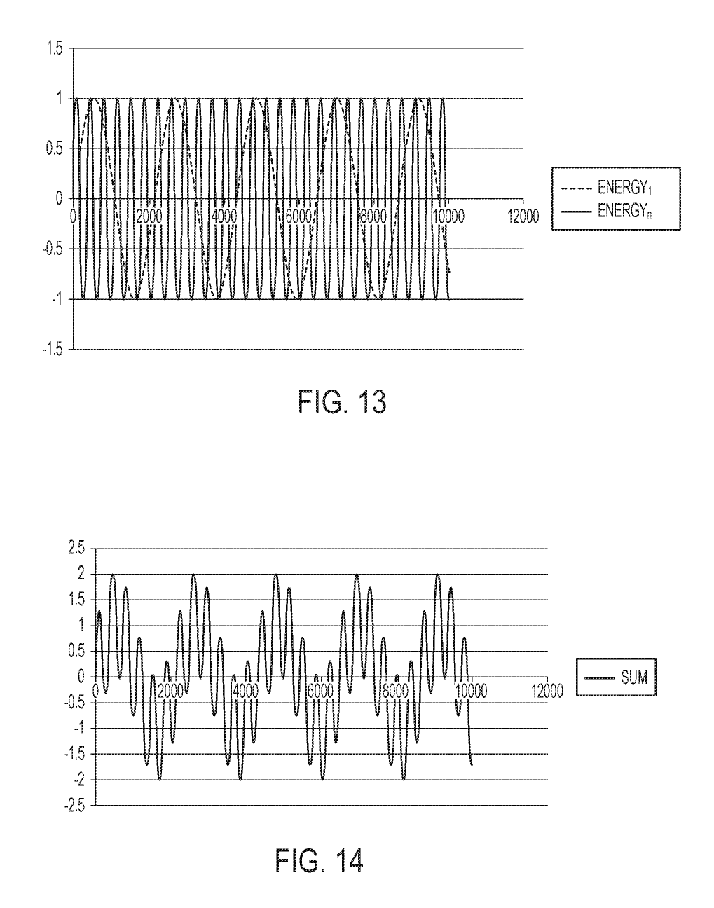

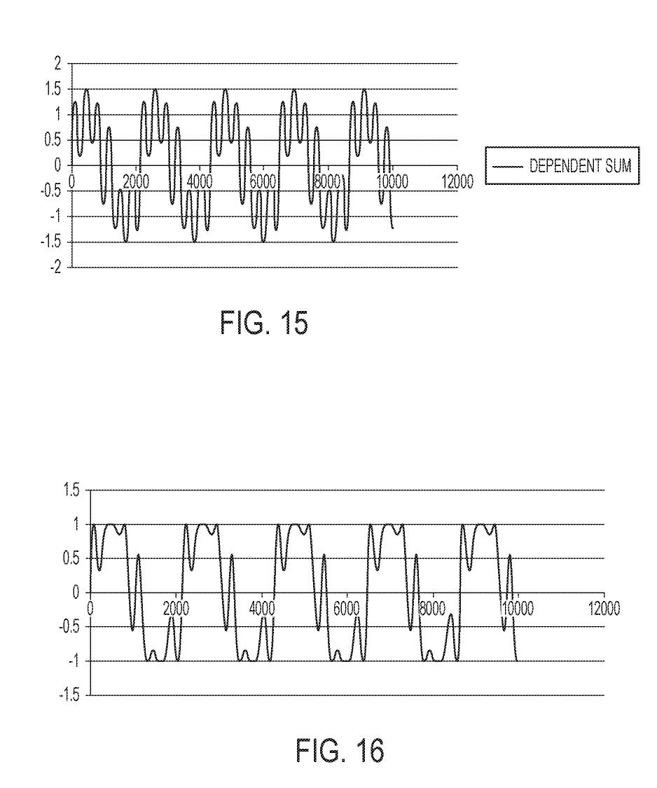

Examples of waveforms representing energy for delivery from a generator are illustrated in FIGS. 13-17. FIG. 13 illustrates an example graph showing first and second waveforms representing first and second energy modalities (such as, for example, ultrasonic, bipolar or monopolar RF, reversible and/or irreversible electroporation, and/or microwave energy modalities). As shown in FIGS. 13-17, one waveform, indicated as ENERGYₙ on the legend, represents, for example, an RF signal at a 330 kHz frequency and another waveform, indicated as ENERGY₁ in the legend, represents, for example, an ultrasonic signal at a 55 kHz frequency. The time and amplitude scales shown in FIG. 13 are normalized. FIG. 14 illustrates an example graph showing the sum of the waveforms of FIG. 13. The peaks from the output are twice the amplitude of the original signals shown in FIG. 13. This can cause problems with the output, such as distortion, saturation, clipping of the output, or stresses on the output components. Thus, the management of the at least one waveform that has multiple treatment components is an important aspect of the at least one output port generator. There are a variety of ways to achieve this management. In one form, one of the outputs or signals can be dependent on the peaks of the other output or signal. For example, as shown in FIG. 15, the RF output depends on the peaks of the ultrasonic output such that the RF output is reduced when an ultrasonic peak is anticipated. As shown in the example graph in FIG. 15, the peaks have been reduced from 2 to 1.5. In another form, one of the outputs or signals is a function of the other output or signal. For example, as shown in FIG. 16, the RF waveform is a function of the ultrasonic waveform. This provides a hard limit on the amplitude of the output. As shown in FIG. 16, the ultrasonic waveform is extractable as a sine wave while the RF waveform has distortion but not in way to affect the coagulation performance of the RF waveform.

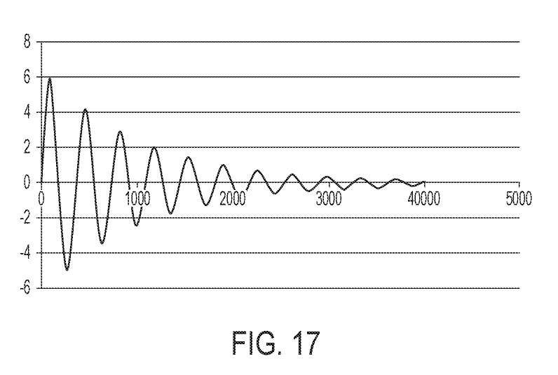

A variety of other techniques can be used for compressing and/or limiting the waveforms. It should be noted that the integrity of the ultrasonic wave and the integrity of the RF waveform may differ as long as the RF waveform has low frequency components for safe patient levels so as to avoid neuro-muscular stimulation. In another form, the frequency of an RF waveform can be changed on a continuous basis in order to manage the peaks of the waveform. Waveform control for more complex RF waveforms, such as a coagulation-type waveform, as illustrated in FIG. 17, may be implemented with the system.

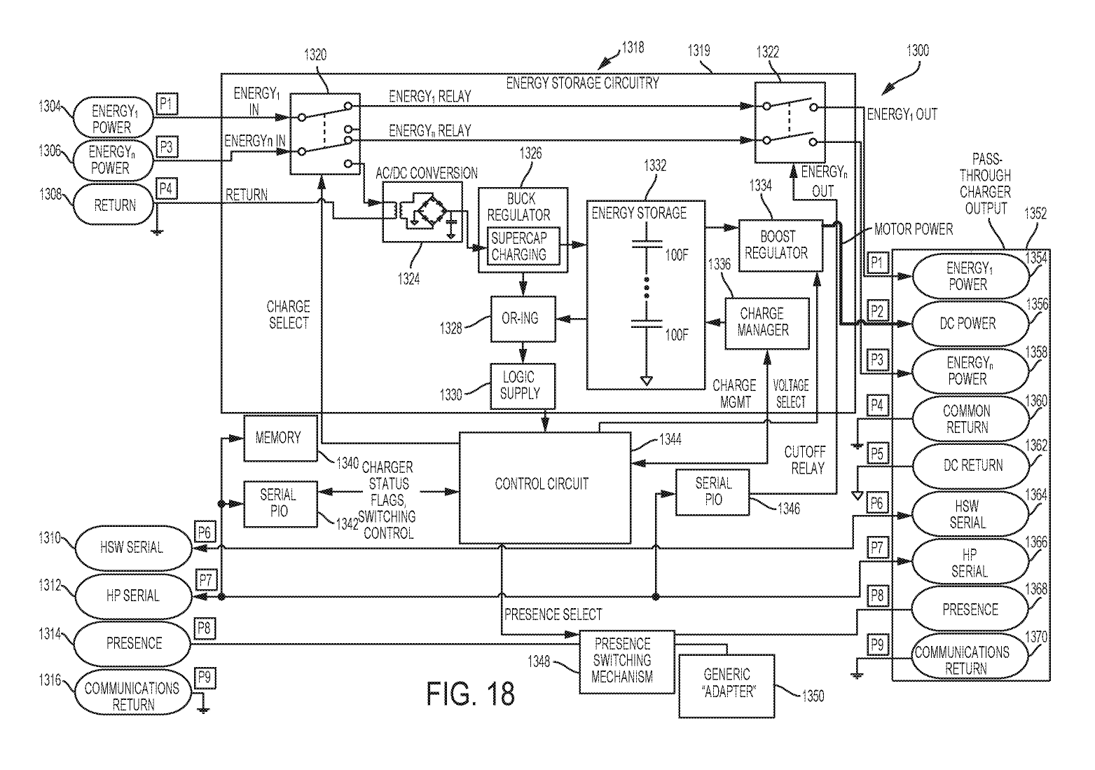

In another aspect of the disclosure, a device can be coupled to the generator that is configured to provide a DC output for various other devices and/or instruments. In one aspect, a generator can be configured to couple to a device, such as an adapter that operates on charging technology from the generator to provide a DC output. FIG. 18 shows a diagram of an electrosurgical system 1300 that includes an adapter 1318 that electrically couples to a generator, such as any of the generators described herein, and utilizes charging technology from the generator to generator DC output for various uses.

As shown in FIG. 18, a generator is coupled to the adapter 1318. The generator provides various inputs to the adapter 1318, including a first energy modality power input 1304, a second energy modality power input 1306, and a common return 1308. As disclosed herein, various energy modalities include ultrasonic, bipolar or monopolar RF (electrosurgical), irreversible and/or reversible electroporation, and/or microwave based surgical instruments, among others. The generator also includes communication interfaces, including a handswitch serial interface 1310 and an handpiece serial interface 1312, a presence element 1314, and a communications return 1316. In one aspect, the presence element 1314 may be a resistor. The generator can use the handswitch and handpiece serial interfaces 1310, 1312 to communicate with the adapter 1318.

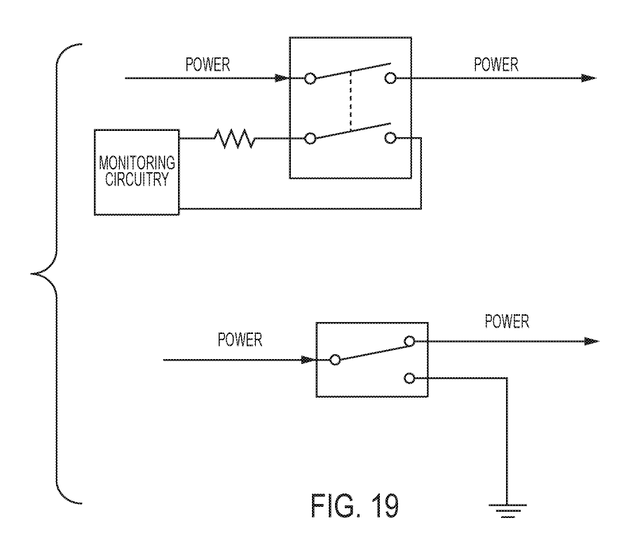

The adapter 1318 comprises energy storage circuit 1319 and control circuit 1344. The energy storage circuit 1319, shown in more detail in FIGS. 19-21, comprises switching mechanisms such as relays 1320, 1322, an AC/DC converter 1324, a buck regulator 1326, a logic supply 1330, an energy storage device 1332, a boost regulator 1334, a charge manager 1336, and various other circuit. The control circuit 1344 may comprise a processor, a field programmable gate array FPGA, CPLD, PLD, microcontroller, DSP, and/or an ASIC, for example, and can communicate with an adapter memory 1340 (e.g., a NVM such as an EEPROM), a serial PIO 1342, a presence switching mechanism 1348, and a generic adapter 1350.

The adapter 1318 also includes an adapter output 1352 for delivering energy to one or more surgical instruments coupled thereto. The adapter output 1352 includes a first energy modality power output 1354 (ENERGY₁ POWER), a DC motor power output 1356, and a second energy modality power output 1358 (ENERGY₁ POWER). Up to n energy modality power outputs are contemplated, where n is a positive integer greater than 2. The adapter output 1352 also include a handswitch serial protocol interface 1364, an handpiece serial protocol interface 1366, and a presence interface 1368. The adapter output 1352 includes various grounds, including a common return 1360, a DC motor power return 1362, and a communications return 1370. The relay 1322 is coupled to the energy modality energy outputs 1354, 1358 of the adapter outputs 1352. A presence switching mechanism 1438 is coupled to the presence interface 1368 and is used for detecting the presence of a surgical device coupled to the adapter.

The system is configured to provide the ability to isolate energy passing to the tissue to prevent tissue damage. As shown in FIG. 19, the capacitors/batteries are charged without any energy passing to the tissue. The relay 1320 may be coupled to a local ground to act as a power return. Thus, the relay provides isolation to the patient to prevent the battery or a supercapacitor from draining to the patient. This isolation of the patient can be achieved using a variety of devices, including but not limited to an opto-isolator.

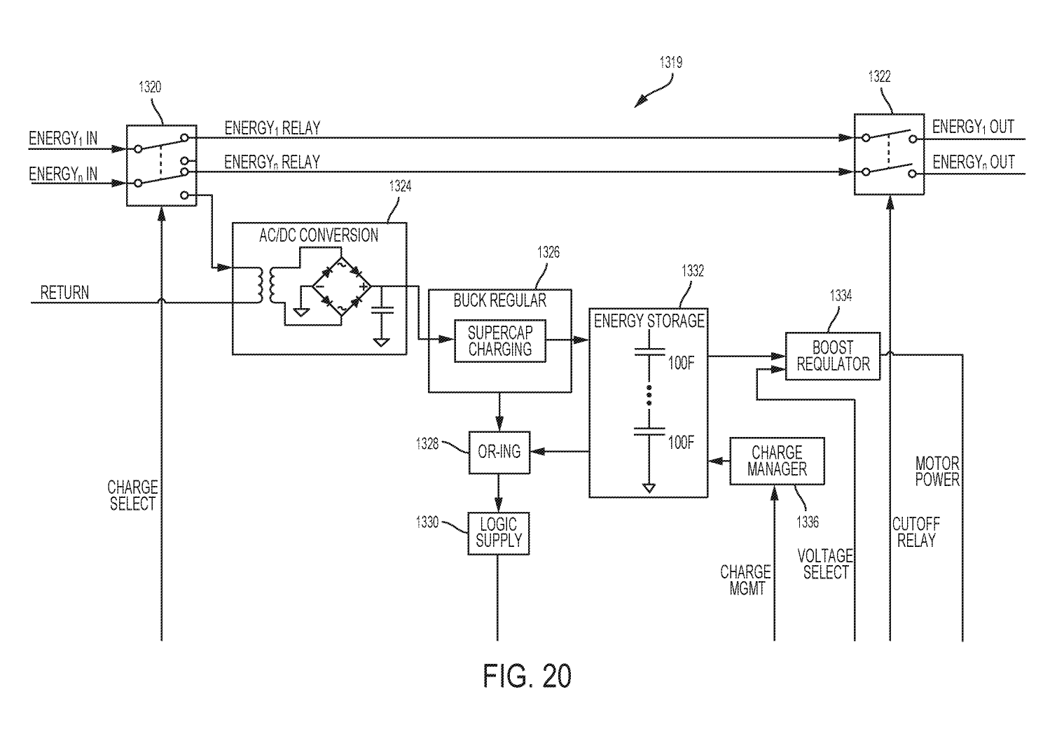

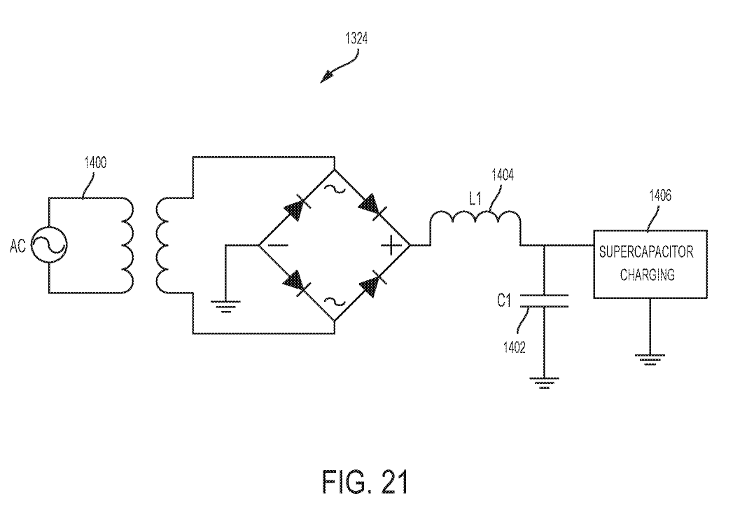

Referring to FIG. 20, the energy storage circuit 1319 includes an AC/DC converter 1324. In one aspect, the AC/DC converter 1324 is a high power AC/DC converter, as shown in FIG. 21. A transformer 1400 is configured to decrease the voltage to a useable level, and a capacitor 1402 is configured to smooth out the rectified voltage. The AC/DC converter 1324 can also optionally include an inductor 1404 that is configured to reduce the peak current at the output of the AC/DC converter 1324.

The AC/DC converter 1324 is configured to communicate with the buck regulator 1326, shown in FIG. 20, that includes one or more supercapacitors. The supercapacitors can be in either a series or parallel configuration. In a series configuration, the supercapacitors can charge faster, the charging current is provided to the capacitors in series, and there is a higher voltage output than if the supercapacitors were in parallel. In a parallel configuration, charging is slower due to the charging current being split between the supercapacitors in parallel. With either configuration, an output boost stage is used.

The following are example equations relating to the useable energy produced from a series of supercapacitors:

$${E}_{useable} := \frac{1}{2} \cdot {C}_{eff} \cdot ( {V}_{charge}^{2} - {V}_{final}^{2} ) \cdot {η}_{{post}_{caps}}$$

$${C}_{eff} = \frac{2 \cdot {E}_{useable}}{( {V}_{charge}^{2} - {V}_{final}^{2} ) \cdot {η}_{{post}_{caps}}}$$

For example, when E_{useable} is 500 J, V_{charge} is 10.26V, V_{final} is 6.0V, and ηₚₒₛₜ_{₁₃} _{caps} is 80%, then the minimum C_{eff} is 18 F. When this example includes four capacitors in a series, the capacitor value for individual capacitors, C_{single,} is the value for C_{eff} multiplied by four. Thus, in this example, the minimum capacitor value is 72 F. It can be seen that four series 100 F capacitors may be sufficient, even after aging to 80% of their initial value. Using the above equations with V_{charge} as 10.26V, _{final} as 6.0V, ηₚₒₛₜ_{_}_{caps} as 80%, C_{eff} as 20 F, which is 80% of the initial value, a useable energy of 554 J should be available, even at the end of the supercapacitor lifetime.

Using different numbers of supercapacitors in series changes various features relating to the supercapacitors, including the charging time. For example, the use of five supercapacitors in series can shorten the charging time of the supercapacitors by taking advantage of the relationship of the energy stored in the capacitor. This can achieve, for example, an approximately 0.5 s reduction in the charging time. However, having just four capacitors in series can provide the energy, and the size of the four supercapacitors can be large. The use of a fifth supercapacitor in series can push the stack voltage to over 12V, requiring an additional component, such as a buck and boost regulator, to provide a DC source.

Another factor relating to the use of rapidly-charging supercapacitors is the power available to charge them. The amount of current and power for charging the supercapacitors varies during the charging cycle of the supercapacitors. The charging scheme can be broken down into three regions, in which the charge current is constant, the charge power is constant, and when the supercapacitor voltage is constant when the charging is complete. At the beginning of the charging cycle, high current is provided, while high power becomes is provided as the voltage of the supercapacitor increases. When charging the supercapacitors to a constant current, eventually the product of voltage and current can exceed the power provided by the generator. In order to accommodate the limited power provided by the generator, the is reduced. This reduction in current can slow down the charging rate of the supercapacitors.

In one aspect, an example energy storage cycle can be used for charging the supercapacitors without overcharging them. A charge select relay is configured to be connected to the energy storage device to allow an adapter with no DC power supply to be charged without direct communication to the microcontroller. The cutoff relays will normally be open, and not conducting current to the surgical device. This ensures that in the event that the charge select relay fails and sticks in a position that is outputting power to the patient, the initial power output from the generator will not reach the patient without the event of a double failure. Upon connection to the generator, or startup of the generator, the generator is configured to identify the adapter through an NVM (e.g., EEPROM) and presence element. The generator is configured to load parameters from the serial NVM of the adapter. After identification of the adapter, the generator is configured to begin outputting power to charge the adapter. In one aspect, this can be done without acknowledgement from the adapter. After the initially applying RF energy to the adapter, the logic supply is configured to power up, allowing the generator to communicate with the adapter as the supercapacitors of the adapter being to charge. In one aspect, during this phase of the charging process, the charge manager will not have enough voltage to report any charge status or capacitor health information to the microcontroller. A timer is configured to wait for a signal from the adapter that its logic devices have enough power to function. This allows for regular communication between the adapter and the generator. Until this signal is received, the generator is configured to assume that the adapter is faulty. Once communication has been established between the adapter and the generator, the generator can configure the adapter with settings for its DC power output, and any other start up parameters.

The supercapacitors are configured to begin charging in the constant current mode. The supercapacitors can begin reporting charge status and capacitor health to the generator once the supercapacitors have charged to a predetermined value. For example, the supercapacitors can begin reporting after they have charged for one or two seconds. The generator is configured to request charge status reports from the adapter 1318. The adapter 1318 is configured to respond to these requests with response packets, notifying the generator of the charge level of the adapter. Before the power limit on the generator is reached, the buck regulator 1326 that charges the supercapacitors will switch from the constant current charging mode to the constant power mode by setting an input current limit.

Based on the charge status reports from the adapter 1318, the generator is configured to recognize when the adapter 1318 is fully charger, or is at least charged to an acceptable level for activation. The generator can then make decisions regarding the switch between outputting power to the adapter 1318 and delivering power for the activation of a surgical instrument coupled thereto. If the generator makes the switch, the generator is configured to wait for an acknowledgement that the request was received and carried out by the relays. Regular communication between the adapter 1318 and the generator is configured to continue such that the generator can output power for activation while monitoring the status of the adapter 1318. When the activation period ends, the generator can begin to recharge the adapter 1318.

Various types of information can be communicated between the adapter and the generator. For example, the adapter 1318 can communicate its energy storage status, the current charge level, and indicate if enough energy is available for activation. The adapter can also coordinate detection of an attached surgical instrument, and/or control the destination of the output power from the generator and direct the output power to either the charging circuit of the adapter 1318 or to the attached surgical instrument. The adapter 1318 is also configured to switch the DC voltage supply between a plurality of pre-set voltage values, for example, 12V, 24V, or 36V. The DC output value can be set by the generator, via relays, or using a microcontroller, CPLD, or serial with supporting circuit.

Switching between delivering energy to the adapter storage device or the surgical device is achieved through the use of relays. A charge select relay is configured to route energy between energy storage in the adapter 1318 and the surgical instruments. A cut-out relay is configured to act as a safeguard to prevent accidental energy output. The cut-out relay can include various features to prevent this accidental output. For example, the cut-out relay can be a force guided relay, also known as a captive contact or safety relay, such that it mechanically links the contacts to prevent the contacts from being in opposite states so that they can switch as a group. If one contact fuses, the remaining contacts will not be able to switch, which allows for monitoring of the relay state since a monitor circuit on one pole of the relay can positively determine the state of the other poles. In one aspect, the relay will switch states when the RF or ultrasonic output is off, but during a fault condition the relay switch may be under the full load.

The output of the relay varies depending on the input energy. In one aspect, a ultrasonic output can drive at least 200 watts at up to 420 VAC and 750 mA, and the RF output can drive at least 130 watts at up to 100 VAC and 3.5 A. Relays with the form factor can max out around 400-450 VAC switching capacity at no load. As the load increases, there is a derating that is applied to the switching capacity. One example relay that can be used is the SR4 D/M relays that can switch up to 400 VAC, but at small currents. In one aspect, relays rated with a mechanical lifetime that is over 1 million cycles can be used. When switching under a load, the electrical life is on the order of 30,000-100,000 cycles before the contacts reach 1 ohm DCR. That level of resistance can increase the power losses and detune the compensation of the generator output. The wear of the relay contacts can be reduced by switching the relays when no load is being driven.

In one aspect, the adapter has a logic voltage, for example, 3.3V, and 5V to drive the charging relay and the cut-off relay. The logic supply is derived from either the DC link during charging of the energy storage circuit 1319 of the adapter 1318 or from the energy storage device when the generator is delivering energy to the surgical instruments. For example, the voltage can be stepped down to 5V using the buck converter, and this can be used to drive the relays. The 5V can be reduced to 3.3V by a low drop-out (LDO) regulator for use by the microcontroller or the CPLD.

The adapter can also have the capability to identify a device that is coupled thereto, for example a surgical instrument, and this information can be communicated to the generator from the adapter. As explained above, the generator identifies the adapter 1318, which occurs one time. After the initial identification of the adapter, the generator is aware of its presence until the adapter is disconnected from the generator or the generator is powered down. The amount of time it takes the generator is identify the adapter is substantially the same as it would take the generator to identify any other device, such as a surgical instrument, connected thereto. After the adapter is identified by the generator, any surgical instrument coupled thereto also is identified, and this identification takes substantially the same amount of time as identifying the adapter. Thus, when an adapter is connected between the generator and a surgical instrument, it takes the generator approximately double the amount of time to identify a surgical instrument as it requires additional time to initially identify the adapter itself. In addition to the time to identify the adapter and the surgical device, time may be required for switching between the presence elements, and can depend on the switching speed of the hardware, the processing speed of the microcontroller or CPLD, or the speed of the serial protocol used for communication between the generator and the microcontroller or CPLD via the serial PIO device. Additional time also may be required to recognize and process data from surgical instruments as the surgical device NVM (e.g., EEPROM), the adapter NVM, and the PIO device are using the same handpiece serial line. Various techniques can be used to identify the surgical instruments coupled to the adapter. For example, identification can be achieved using the NVM of the surgical instrument, a bar code on the surgical instrument, a resistor ID, or any other method that can be employed for identification.

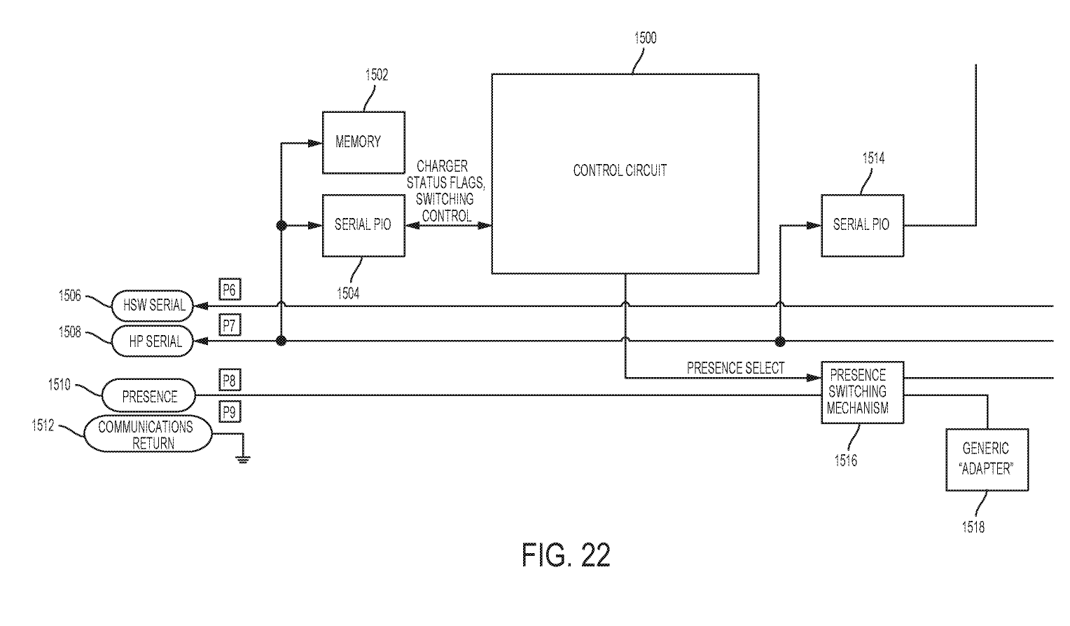

FIG. 22 illustrates one aspect of a scheme for identification of an adapter by a generator. As shown, a handswitch (HSW) serial line 1506, a handpiece (HP) serial line 1508, a presence 1510, and communications return 1512 terminals are provided. A serial PIO circuit 1504 is coupled to the handpiece serial line 1508 communicates charger status flags, and switching control to the control circuit 1500. Other serial PIO circuits 1514 may be coupled to the handpiece serial line 1508. A presence switching mechanism 1516 is coupled to the control circuit 1500 and the presence serial line 1510. A generic adapter 1508 is coupled to the presence switching mechanism 1516. The adapter is configured to include a unique presence element 1510 to identify itself to the generator. In one aspect, the presence element 1510 may be a resistor. The presence element 1510 is connected to a presence line. The generator can identify the adapter using a combination of the presence element 1510 and a memory 1502 (e.g., a NVM such as an EEPROM). The adapter can contain a constant current source and an ADC to read the presence value of a surgical device connected thereto. The ADC can be connected directly to a control circuit 1500 or to one or more of the serial IO expanders. The control circuit 1500 may comprise a processor, FPGA, CPLD, PLD, microcontroller, DSP, and/or an ASIC, for example. Thus, communications from the generator to a serial device is utilized to transmit ADC data to the generator. This scheme can be scalable and extend to multiple adapters connected in series. The adapters read the presence of the next adapter on its ADC, and the ADC values can be communicated over serial IO expanders on the handpiece serial line using the handpiece (HP) serial interface 1508. In one aspect, the presence element (HRS70.2 and HRS70.3) of the generator can state that the generator reads presence resistances in the range of 0-310 ohms at an accuracy of+/−5% (or 10 ohms). To meet these requirements, a 10-bit or 12-bit ADC can be provided for a variety of reasons, including to accommodate a band at the top of the range of the ADC for instances when no presence element is connected. More than one serial device may be employed to connect directly to the ADC. This scheme can allow for substantially simultaneous presence readings of the series adapters, but can also employ an ADC and a current source external to the generator.

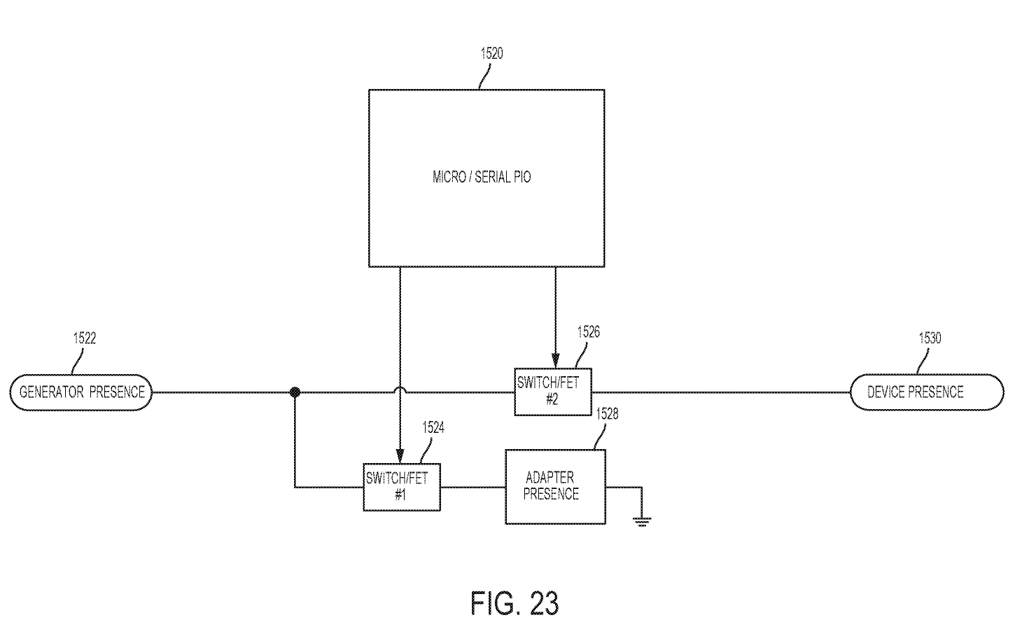

FIG. 23 illustrates another aspect of a scheme for identifying an adapter in which a switching scheme is used to detect changes in presence. Switches 1524, 1526 are coupled between the generator presence 1522 and the device presence 1530 terminals. The first and second switches 1524, 1526 also are coupled to a Micro/Serial PIO control circuit 1520. The Micro/Serial PIO control circuit 1520 controls the operation of the switches 1524, 1526. The first switch 1524 is coupled to an adapter presence circuit 1528. The generator is configured to read the ADC values and use this information to detect changes in presence. The serial expanders would be used to control the switching of the resistors. Thus, in this scheme, the generator identifies the pass though charger and then uses the switch relays to look for and identify any additional devices. Referring to FIG. 23, when the generator is powered on, a switch 1524 is closed and a switch 1526 is open. The generator is configured to begin presence detection in order to identify an adapter 1528, such as an adapter, connected thereto, which is identified by its presence element and an NVM in order to identify the adapter. The generator then closes the switch 1524 and the switch 1526 such that they are connected in parallel. The generator is configured to monitor for any changes in resistance. If the resistance changes to open, the adapter may have been unplugged from the generator, and if the resistance changes values, a new device may have been connected. If so, the generator is configured to open the switch 1524 and begin the process of identifying the device. This scheme allows the generator to recursively identify the connected devices, including an adapter and any surgical devices. After the generator has identified the devices, the parallel resistance of the identified device is monitored for resistance changes. The generator uses existing monitoring hardware and can detect changes by monitoring at least one presence value.

Communication options between the generator and adapter can take a variety of forms, but should accommodate and co-exist with the existing surgical device communications for a wide range of surgical devices and the available number of signal wires. In one aspect, the adapter can utilize the handpiece signal wire and/or the handswitch signal wire.

The handswitch wire connected to the handswitch serial interface 1310 shown in FIG. 18 is used by the generator to communicate with the surgical devices coupled thereto. For example, it can detect a button being pressed on a surgical device and can be used to communicate with the NVMs of the surgical devices. In one aspect, it is a current controlled signal+/−15 mA with a voltage cap of 5V. It can operate at a higher voltage than the handpiece wire line, and can communicate at higher serial speeds and is less affected by noise than the handpiece wire line. The handswitch wire line can be complex, an) circuits can use it in a hybrid manner for NVM and switching communications. The NVM communicate does not occur while surgical device buttons are being monitored, and vice versa.

The handpiece wire connected to the handpiece serial interface 1312 shown in FIG. 18 is used by the generator to communicate with the NVMs on ultrasonic headpieces. The handpiece wire is dedicated to NVM communication and is powered by a 3.3V pull-up resistor. Communications on the handpiece wire are slightly slower than on the handswitch wire as it is a 3.3V bus rather than a 5V bus, but it is also less burdened than the handswitch wire. The handpiece wire is queried during a change of surgical device presence, and most of the traffic on the bus is during NVM reads and writes.

In one example communication method utilizing the handpiece wire, a multi-channel expandable switch is connected to the handpiece serial line to form a basis for the communication. The 8-channel IO expanders can be parasitically powered and supported, and are capable of reads and writes. The output from the serial IO expanders includes a high impedance output to act as a current sink. The serial IO expanders cannot source current for any of the attached devices such that the pass though charger needs its own power supply.

Another example communication method involves a smart device in the adapter that directly interfaces with the serial bus. A further example communication method involves using the serial interface as a hybrid interface such that a different high speed protocol, such as a half-duplex UART, is used to communicate with the adapter, and the serial protocol is used to talk to the handpiece. If the serial communications speed is fast enough, for example on the order of 100 kbits/s, then it should be unlikely that the serial device should see a reset pulse for all the devices to communicate on the bus and the serial devices, and the UART bus do not interfere with one another.

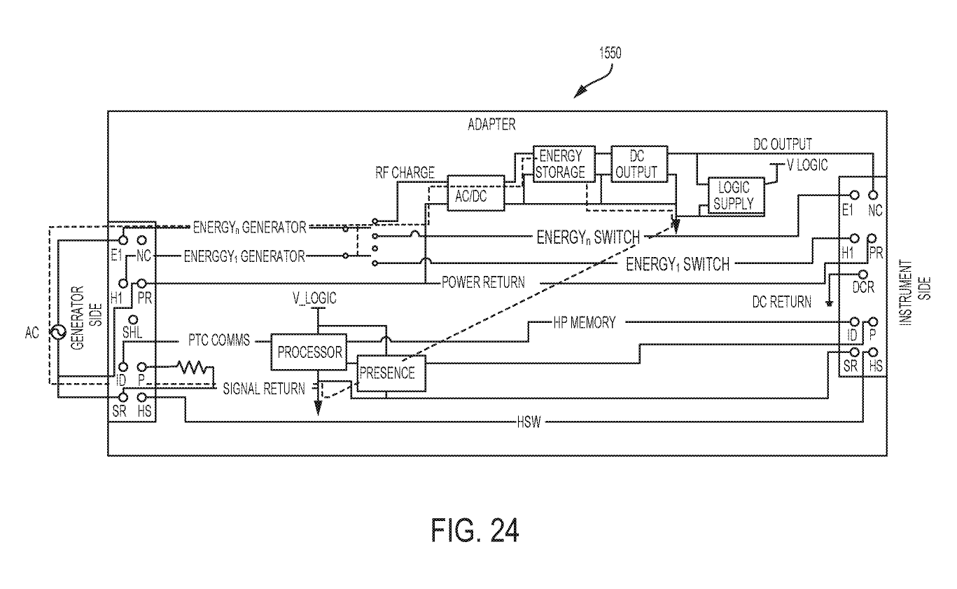

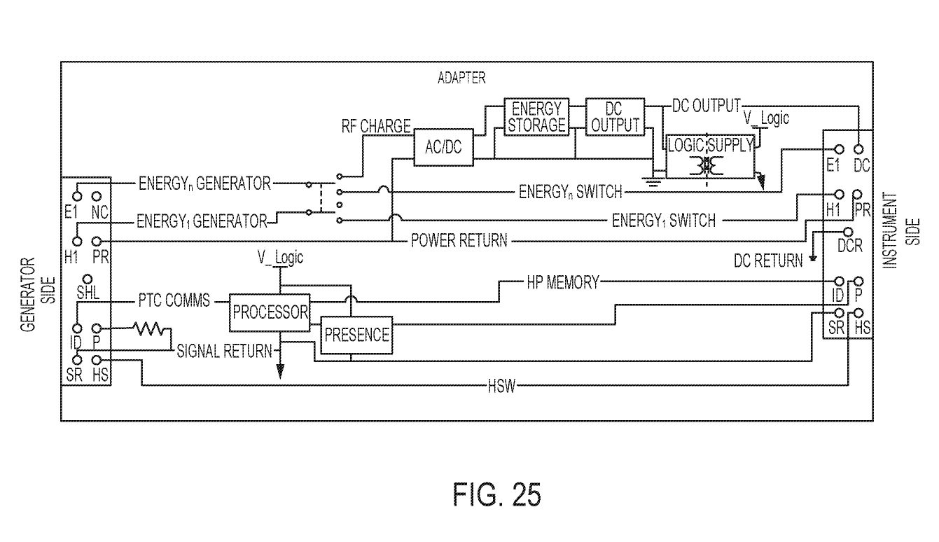

The adapter also includes functional isolation features to prevent charging currents from returning on the communication ground and to provide a common potential for the communication circuit to the generator. FIG. 24 illustrates an adapter 1550 showing charging currents returning on a communication ground. With the logic supply sharing the ground of an AC/DC converter, the charging current will likely return on the signal return path, which can cause signal quality issues if the charging current is high. To prevent this, an isolated supply for the logic circuit can be used, as shown in FIG. 25. This isolated supply allows for separation of the grounds. The microcontroller also can be isolated (but is not shown in FIG. 25).

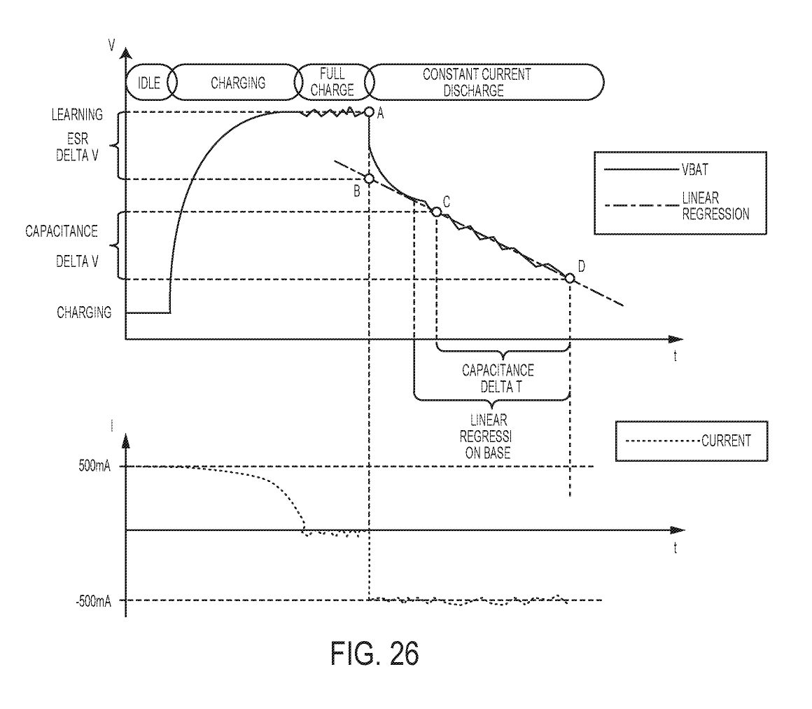

The charging performance of a pass through charge may be monitored, including long term and short term performance. A supercapacitor charge management circuit can support the detection of performance degradation, including capacitance value and the equivalent series resistance (ESR) of the supercapacitors. The industry standard declares the performance criteria to be <20% reduction in capacitance value and <100% increase in ESR. The charge management IC will periodically execute a learning cycle during which it discharges the supercapacitors at a constant current and records the voltages at specific time intervals. FIG. 26 illustrates the determination of the capacitance and ESR. Capacitance is calculated using

$$C = I * \frac{( T [ D ] - T [ C ] )}{( V [ C ] - V [ D ] )}$$

ESR is calculated using

$$ESR = \frac{( V [ A ] - V [ B ] )}{I}$$

The generator and adapter can also include features to dissipate the energy stored in an adapter upon shut down of a generator. It could be problematic to leave energy stored in the adapter because that energy can potentially discharge, for example, to a patient. The energy in the adapter may be dissipated when a surgical instrument is unplugged while the adapter is fully charged as well. To achieve this energy dissipation, in one aspect a timing-controlled, energy dissipation resistor can be used. It can be a ceramic, wirewound, or load resistor, and can be used to quickly dissipate the energy. In another aspect, a diode can be used to indicate whether or not there is energy stored in the adapter, and can be used to slowly dissipate the energy.

There can also be a certain amount of heat that accumulates during use of a generator and an adapter, for example, from electrical losses. After shutdown, unplugging, or simply from normal use, thermal effects within the system can have an undesired effect. Various devices can be used to monitor the heat in the adapter. In one aspect, a thermal sensor can be added to the adapter and can monitor the heat of the device. The thermal sensor can be configured to shut down the adapter in the event that the heat is higher than a certain designated threshold. The generator would register this as a fault of the adapter. In another aspect, a heat sink can be used to prevent overheating during charging or driving of a DC load.

Methods can also be employed to allow an adapter to charge during activation of a surgical device coupled thereto. In one aspect, time slice charging can be used to charge the adapter while a surgical device is in use. This would allow a small percentage of the activation energy being used to drive a surgical device to be used for charging the adapter. In another aspect, energy leaching can be used to charge the adapter off of RF energy while activating a surgical device. RF antennas and inductive charging allow for the slow charging of capacitors or batteries. For example, if an algorithm being used by a surgical device does not require the full energy output of the generator, the generator can provide energy to the surgical device while providing any extra energy that the generator can output but that is not required by the algorithm to the adapter for charging.

The surgical instruments described herein can also include features to allow the energy being delivered by the generator to be dynamically changed based on the type of tissue being treated by an end effector of a surgical instrument and various characteristics of the tissue. In one aspect, an algorithm for controlling the power output from a generator, such as generator 1002, that is delivered to the end effector of the surgical instrument can include an input that represents the tissue type to allow the energy profile from the generator to be dynamically changed during the procedure based on the type of tissue being effected by the end effector of the surgical instrument.

According to the present disclosure, a generator, such as generator 1002 described herein, may be configured to provide a number of wave shapes, in the form of waveform signals, to a surgical instrument so that the surgical instrument may apply a therapy to tissue.

The generator may generate its output waveform digitally, which means the wave shape can be digitized by a number of points which are stored in a table. The points may be stored in the table with a Field Programmable Gate Array (FPGA). In one aspect, the wave shape is digitized into 1024 points. The generator software and digital controls may command the FPGA to scan the addresses in this table which in turn provides varying digital input values to a DAC that feeds a power amplifier. The addresses may be scanned according to a frequency of interest. Using such a table enables generating various types of wave shapes that can be fed into tissue or into a transducer, an RF electrode, multiple transducers simultaneously, multiple RF electrodes simultaneously, or a combination of RF and ultrasonic instruments. Furthermore, multiple wave shape tables can be created, stored, and applied to tissue for a generator.

The waveform signal may be configured to control at least one of an output current, an output voltage, or an output power of an ultrasonic transducer and/or an RF electrode. Further, where the surgical instrument comprises an ultrasonic components, the waveform signal may be configured to drive at least two vibration modes of an ultrasonic transducer of the at least one surgical instrument. Accordingly, a generator may be configured to provide a waveform signal to at least one surgical instrument wherein the waveform signal corresponds to at least one wave shape of a plurality of wave shapes in a table. Further, the waveform signal provided to the two surgical instruments may comprise two or more wave shapes. The table may comprise information associated with a plurality of wave shapes and the table may be stored within the generator. In one aspect or example, the table may be a Direct Digital Synthesis (DDS) table, which may be stored in an FPGA of the generator. The table may be addressed by anyway that is convenient for categorizing wave shapes. According to one aspect, the table, which may be a DDS table, is addressed according to a frequency of the waveform signal. Additionally, the information associated with the plurality of wave shapes may be stored as digital information in the table.

In an aspect, the generator may comprise a Digital-to-Analog Converter (DAC) and a power amplifier, the DAC is coupled to the power amplifier such that the DAC provides digital input values to the power amplifier associated with a wave shape of the plurality of wave shapes for the waveform signal.

Further, the generator is configured to provide the waveform signal to at least two surgical instruments simultaneously. The generator also may be configured to provide the waveform signal, which may comprise two or more wave shapes, via at least one output channel to the two surgical instruments simultaneously. The generator may output the waveform signal having multiple wave shapes to at least one or multiple surgical instruments. For example, in one aspect the waveform signal comprises an ultrasonic signal, an RF signal, and/or a combination of both. In addition, a waveform signal may comprise a plurality of ultrasonic signals, a plurality of RF signals, and/or a combination of a plurality of ultrasonic signals and a plurality of RF signals.

In addition, a method of operating a generator according to the present disclosure comprises generating a waveform signal and providing the generated waveform signal to at least one surgical instrument, where generating the waveform signal comprises reading waveform signal information from a table comprising information associated with a plurality of wave shapes. The generated waveform signal corresponds to at least one wave shape of the plurality of wave shapes of the table. Furthermore, providing the generated waveform signal to the at least one surgical instrument may comprise providing the waveform signal to at least two surgical instruments simultaneously.

A generator as described may allow for the creation of various types of DDS (Direct Digital Synthesis) tables within a generator FPGA. Examples of wave shapes in the RF/Electrosurgery tissue treatment field that may be accomplished by such a generator are as follows: High crest factor RF signals (which may be used for surface coagulation in RF mode); Low crest factor RF signals (which may be used for deeper tissue penetration); and waveforms that promote efficient touch-up coagulation. The generator also may allow for the creation of multiple wave shape tables and, on the fly, be able to switch between the wave shapes based on tissue effect. Switching may be based on tissue impedance and/or other factors.

A generator as described also may allow for, in addition to the traditional sine wave shape, wave shape(s) that maximizes the power into tissue per cycle (i.e. trapezoidal or square wave). It also may provide wave shape(s) that are synchronized in such way that they make maximizing power delivery in the case RF and ultrasonic signals are driven and a waveform that drives both ultrasonic and RF therapeutic energy simultaneously while maintaining Ultrasonic frequency lock (provided that the circuit topology which enables simultaneously driving RF and Ultrasonic is utilized). Further, custom wave shapes specific to instruments and their tissue effects can be stored in generator non-volatile memory or in instrument NVMs and be fetched upon instrument connection to the generator.

While various details have been set forth in the foregoing description, it will be appreciated that the various aspects of the serial communication protocol for medical device may be practiced without these specific details. For example, for conciseness and clarity selected aspects have been shown in block diagram form rather than in detail. Some portions of the detailed descriptions provided herein may be presented in terms of instructions that operate on data that is stored in a computer memory. Such descriptions and representations are used by those skilled in the art to describe and convey the substance of their work to others skilled in the art. In general, an algorithm refers to a self-consistent sequence of steps leading to a result, where a “step” refers to a manipulation of physical quantities which may, though need not necessarily, take the form of electrical or magnetic signals capable of being stored, transferred, combined, compared, and otherwise manipulated. It is common usage to refer to these signals as bits, values, elements, symbols, characters, terms, numbers, or the like. These and similar terms may be associated with the appropriate physical quantities and are merely convenient labels applied to these quantities.

Unless specifically stated otherwise as apparent from the foregoing discussion, it is appreciated that, throughout the foregoing description, discussions using terms such as “processing” or “computing” or “calculating” or “determining” or “displaying” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

It is worthy to note that any reference to “one aspect,” “an aspect,” “one form,” or “an form” means that a particular feature, structure, or characteristic described in connection with the aspect is included in at least one aspect. Thus, appearances of the phrases “in one aspect,” “in an aspect,” “in one form,” or “in an form” in various places throughout the specification are not necessarily all referring to the same aspect. Furthermore, the particular features, structures or characteristics may be combined in any suitable manner in one or more aspects.

Some aspects may be described using the expression “coupled” and “connected” along with their derivatives. It should be understood that these terms are not intended as synonyms for each other. For example, some aspects may be described using the term “connected” to indicate that two or more elements are in direct physical or electrical contact with each other. In another example, some aspects may be described using the term “coupled” to indicate that two or more elements are in direct physical or electrical contact. The term “coupled,” however, also may mean that two or more elements are not in direct contact with each other, but yet still co-operate or interact with each other.

It is worthy to note that any reference to “one aspect,” “an aspect,” “one form,” or “an form” means that a particular feature, structure, or characteristic described in connection with the aspect is included in at least one aspect. Thus, appearances of the phrases “in one aspect,” “in an aspect,” “in one form,” or “in an form” in various places throughout the specification are not necessarily all referring to the same aspect. Furthermore, the particular features, structures or characteristics may be combined in any suitable manner in one or more aspects.

Although various forms have been described herein, many modifications, variations, substitutions, changes, and equivalents to those forms may be implemented and will occur to those skilled in the art. Also, where materials are disclosed for certain components, other materials may be used. It is therefore to be understood that the foregoing description and the appended claims are intended to cover all such modifications and variations as falling within the scope of the disclosed forms. The following claims are intended to cover all such modification and variations.

In a general sense, those skilled in the art will recognize that the various aspects described herein which can be implemented, individually and/or collectively, by a wide range of hardware, software, firmware, or any combination thereof can be viewed as being composed of various types of “electrical circuit.” Consequently, as used herein “electrical circuit” includes, but is not limited to, electrical circuit having at least one discrete electrical circuit, electrical circuit having at least one integrated circuit, electrical circuit having at least one application specific integrated circuit, electrical circuit forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuit forming a memory device (e.g., forms of random access memory), and/or electrical circuit forming a communications device (e.g., a modem, communications switch, or optical-electrical equipment). Those having skill in the art will recognize that the subject matter described herein may be implemented in an analog or digital fashion or some combination thereof.

The foregoing detailed description has set forth various forms of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one form, several portions of the subject matter described herein may be implemented via ASIC, FPGA, DSP, or other integrated formats. However, those skilled in the art will recognize that some aspects of the forms disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuit and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative form of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link (e.g., transmitter, receiver, transmission logic, reception logic, etc.), etc.).

The above-mentioned U.S. patents, U.S. patent application publications, U.S. patent applications, foreign patents, foreign patent applications, non-patent publications referred to in this specification and/or listed in any Application Data Sheet, or any other disclosure material are incorporated herein by reference, to the extent not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will be incorporated only to the extent that no conflict arises between that incorporated material and the existing disclosure material.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely example, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In some instances, one or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

In certain cases, use of a system or method may occur in a territory even if components are located outside the territory. For example, in a distributed computing context, use of a distributed computing system may occur in a territory even though parts of the system may be located outside of the territory (e.g., relay, server, processor, signal-bearing medium, transmitting computer, receiving computer, etc. located outside the territory).

A sale of a system or method may likewise occur in a territory even if components of the system or method are located and/or used outside the territory. Further, implementation of at least part of a system for performing a method in one territory does not preclude use of the system in another territory.

Although various forms have been described herein, many modifications, variations, substitutions, changes, and equivalents to those forms may be implemented and will occur to those skilled in the art. Also, where materials are disclosed for certain components, other materials may be used. It is therefore to be understood that the foregoing description and the appended claims are intended to cover all such modifications and variations as falling within the scope of the disclosed forms. The following claims are intended to cover all such modification and variations.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

Various aspects of the subject matter described herein are set out in the following numbered clauses:

1. An apparatus for operating a surgical instrument, comprising: a generator configured to provide an energy signal for treating tissue, the generator comprising one or more ports for delivering the energy signal to one or more surgical instruments configured to treat tissue, the one or more surgical instruments being electrically coupled to the respective one or more ports simultaneously, wherein the energy signal comprises an energy component comprising one or more energy modalities that is delivered through the one or more ports.

2. The apparatus of clause 1, wherein the one or more ports are integrated into the generator.

3. The apparatus of any one of clauses 1-2, wherein the one or more ports are separate from the generator and electrically coupled to the generator.

4. The apparatus of any one of clauses 1-3, wherein the generator is configured to deliver the energy signal to any one of the one or more ports.

5. The apparatus of any one of clauses 1-4, wherein the generator is configured to deliver the energy signal to any combination of the one or more ports.

6. The apparatus of clause 5, further comprising a one ore more electrical measurement components coupled to the one or more ports to measure an electrical quantity delivered from the generator to the one or more surgical instruments coupled to the respective one or more ports.

7. The apparatus of clause 6, wherein the electrical quantity is either a voltage or a current.

8. The apparatus of any one of clauses 1-7, further comprising a current monitor, wherein the generator is configured to monitor the activity of each of the one or more surgical instruments using the current monitor.

9. The apparatus of any one of clauses 1-8, wherein the generator is configured to generate a separate energy signal for each of the one or more surgical instruments.

10. The apparatus of any one of clauses 1-9, wherein the one or more energy modalities comprises any one of ultrasonic component, a bipolar radio frequency (RF) component, a monopolar RF component, a reversible electroporation component, an irreversible electroporation component, or a microwave component, or any combination thereof.

11. A method of operating a surgical instrument, the method comprising: providing, by a generator, an energy signal for treating tissue, the generator comprising one or more ports for delivering the energy signal to one or more surgical instruments configured to treat tissue, the one or more surgical instruments being electrically coupled to the respective one or more ports simultaneously, wherein the energy signal comprises an energy component comprising one or more energy modalities that is delivered through the one or more ports.

12. The method of clause 11, further comprising delivering, by the generator, the energy signal to any one of the one or more ports.

13. The method of any one of clauses 11-12, further comprising delivering, by the generator, the energy signal to any combination of the one or more ports.

14. The method of clause 13, further comprising measuring, by a first and second measurement component coupled to the first and second ports, an electrical quantity delivered from the generator to the one or more surgical instruments coupled to the respective one or more ports.

15. The method of clause 13, further comprising monitoring, by the generator, the activity of the one or more surgical instruments using a current monitor.

16. The method of any one of clauses 11-15, further comprising generating, by the generator, a separate energy signal for the one or more second surgical instruments.

17. The method of any one of clauses 11-16, wherein the one or more energy modalities comprises any one of ultrasonic component, a bipolar radio frequency (RF) component, a monopolar RF component, a reversible electroporation component, an irreversible electroporation component, or a microwave component, or any combination thereof.

18. An apparatus for providing a combined energy modality power output, the generator comprising: an adapter comprising an energy storage circuit, wherein the energy storage circuit comprises a switching mechanism configured to receive at least one combined energy modality power input from a generator and provide one or more combined energy modality power outputs; a communication interface; and a presence interface.

19. The apparatus of clause 18, wherein the adapter further comprises a current, a voltage, or a power monitoring circuit, or any combination thereof.

20. The apparatus of any one of clauses 18-19, wherein the adapter further comprises a control circuit.

21. The apparatus of any one of clauses 18-20, further comprising a presence element to uniquely identify the adapter.

22. The apparatus of any one of clauses 18-21, wherein the communication interface comprises a communications circuit to receive communications to or from the generator.

23. The apparatus of any one of clauses 18-22, wherein the energy storage circuit comprises a storage control component, a storage monitoring component, or a device power monitoring component, or any combination thereof.

24. The apparatus of any one of clauses 18-23, wherein the energy storage circuit comprises a data storage component.

25. The apparatus of any one of clauses 18-24, wherein the adapter is configured to provide a direct current (DC) output.

While several forms have been illustrated and described, it is not the intention of the applicant to restrict or limit the scope of the appended claims to such detail. Numerous variations, changes, and substitutions will occur to those skilled in the art without departing from the scope of the disclosure. Moreover, the structure of each element associated with the described forms can be alternatively described as a means for providing the function performed by the element. Accordingly, it is intended that the described forms be limited only by the scope of the appended claims.

Reference throughout the specification to “various forms,” “some forms,” “one form,” or “an form” means that a particular feature, structure, or characteristic described in connection with the form is included in at least one form. Thus, appearances of the phrases “in various forms,” “in some forms,” “in one form,” or “in an form” in places throughout the specification are not necessarily all referring to the same form. Furthermore, the particular features, structures, or characteristics may be combined in any suitable manner in one or more forms. Thus, the particular features, structures, or characteristics illustrated or described in connection with one form may be combined, in whole or in part, with the features structures, or characteristics of one or more other forms without limitation.

What is claimed is:

1. An apparatus for operating a surgical instrument, comprising:
a generator configured to provide an energy signal for treating tissue, the generator comprising one or more ports for delivering the energy signal to one or more surgical instruments configured to treat tissue, the one or more surgical instruments being electrically coupled to the respective one or more ports simultaneously,
wherein the energy signal comprises an energy component comprising one or more energy modalities that is delivered through the one or more ports.

2. The apparatus of claim 1, wherein the one or more ports are integrated into the generator.

3. The apparatus of claim 1, wherein the one or more ports are separate from the generator and electrically coupled to the generator.

4. The apparatus of claim 1, wherein the generator is configured to deliver the energy signal to any one of the one or more ports.

5. The apparatus of claim 1, wherein the generator is configured to deliver the energy signal to any combination of the one or more ports.

6. The apparatus of claim 5, further comprising a one ore more electrical measurement components coupled to the one or more ports to measure an electrical quantity delivered from the generator to the one or more surgical instruments coupled to the respective one or more ports.

7. The apparatus of claim 6, wherein the electrical quantity is either a voltage or a current.

8. The apparatus of claim 1, further comprising a current monitor, wherein the generator is configured to monitor the activity of each of the one or more surgical instruments using the current monitor.

9. The apparatus of claim 1, wherein the generator is configured to generate a separate energy signal for each of the one or more surgical instruments.

10. The apparatus of claim 1, wherein the one or more energy modalities comprises any one of ultrasonic component, a bipolar radio frequency (RF) component, a monopolar RF component, a reversible electroporation component, an irreversible electroporation component, or a microwave component, or any combination thereof.

11. A method of operating a surgical instrument, the method comprising:
providing, by a generator, an energy signal for treating tissue, the generator comprising one or more ports for delivering the energy signal to one or more surgical instruments configured to treat tissue, the one or more surgical instruments being electrically coupled to the respective one or more ports simultaneously,
wherein the energy signal comprises an energy component comprising one or more energy modalities that is delivered through the one or more ports.

12. The method of claim 11, further comprising delivering, by the generator, the energy signal to any one of the one or more ports.

13. The method of claim 11, further comprising delivering, by the generator, the energy signal to any combination of the one or more ports.

14. The method of claim 13, further comprising measuring, by a first and second measurement component coupled to the first and second ports, an electrical quantity delivered from the generator to the one or more surgical instruments coupled to the respective one or more ports.

15. The method of claim 13, further comprising monitoring, by the generator, the activity of the one or more surgical instruments using a current monitor.

16. The method of claim 11, further comprising generating, by the generator, a separate energy signal for the one or more second surgical instruments.

17. The method of claim 11, wherein the one or more energy modalities comprises any one of ultrasonic component, a bipolar radio frequency (RF) component, a monopolar RF component, a reversible electroporation component, an irreversible electroporation component, or a microwave component, or any combination thereof.

18. An apparatus for providing a combined energy modality power output, the generator comprising:
an adapter comprising an energy storage circuit, wherein the energy storage circuit comprises a switching mechanism configured to receive at least one combined energy modality power input from a generator and provide one or more combined energy modality power outputs;
a communication interface; and
a presence interface.

19. The apparatus of claim 18, wherein the adapter further comprises a current, a voltage, or a power monitoring circuit, or any combination thereof.

20. The apparatus of claim 18, wherein the adapter further comprises at least one control circuit.

21. The apparatus of claim 18, further comprising a presence element to uniquely identify the adapter.

22. The apparatus of claim 18, wherein the communication interface comprises a communications circuit to receive communications to or from the generator.

23. The apparatus of claim 18, wherein the energy storage circuit comprises a storage control component, a storage monitoring component, or a device power monitoring component, or any combination thereof.

24. The apparatus of claim 18, wherein the energy storage circuit comprises a data storage component.

25. The apparatus of claim 18, wherein the adapter is configured to provide a direct current (DC) output.
